(19) United States
(12) Patent Application Publication
(10) Pub. No.: US 2026/0027018 A1
(43) Pub. Date: Jan. 29, 2026

(54) BIODEGRADABLE MICROBEADS WITH ENHANCED OPTICAL EFFECTS

(71) Applicant: Eastman Chemical Company, Kingsport, TN (US)

(72) Inventors: ROBERT DAVID PACE, CHURCH HILL, TN (US); MATT STEPHENS, KINGSPORT, TN (US); DEEPIKA SAINI, KINGSPORT, TN (US); BRAJESH JHA, KINGSPORT, TN (US); JUNG HYUN PARK, JOHNSON CITY, TN (US); LESLIE TAYLOR BAKER, GATE CITY, VA (US); HASSAN ALOMGIR ALI, ANTWERPEN (BE); KENNY RANDOLPH PARKER, AFTON, TN (US)

(73) Assignee: Eastman Chemical Company, Kingsport, TN (US)

(21) Appl. No.: 18/998,908

(22) PCT Filed: Mar. 22, 2024
(86) PCT No.: PCT/US2024/021024

§ 371 Date: Jan. 27, 2025

Related U.S. Application Data
(60) Provisional application No. 63/491,606, filed on Mar. 22, 2023.

(51) Int. Cl. A61K 8/02; A61K 8/73; A61Q 19/00; C08B 3/18; C08L 1/14

(52) U.S. Cl. CPC A61K 8/025; A61K 8/731; A61Q 19/00; C08B 3/18; C08L 1/14; A61K 2800/10; A61K 2800/412; C08L 2201/06

(57) ABSTRACT

Biodegradable microbeads exhibiting superior optical effects are provided. More particularly, biodegradable microparticles formed from biodegradable mixed cellulose esters are provided that exhibit superior optical effects, such as haze transmission, which are highly beneficial when used to produce cosmetic formulations. The produced microbeads also exhibit enhanced solidity, sphericity, and smoothness.

### BACKGROUND

Microbeads are particles that are less than 1 millimeter (mm) in diameter. These particles are sometimes included in consumer products, such as personal care and cosmetic products. Many of these microbead-containing products are designed to be applied and then washed or rinsed from the user's body. When the microbead-containing products are washed or rinsed from the user's body, the particles are flushed down the drain and received at municipal water treatment facilities. In the past, many known microbeads had been formed from plastic or polymeric materials, such as polyethylene, polypropylene, polymethyl methacrylate, nylon, polyurethane, and the like. These materials generally have limited biodegradability. In addition, the small size of the particles limits their ability to be captured at the water treatment facilities, such that the particles may be discharged from the facilities and into larger bodies of water (e.g., rivers, seas, and oceans). Once in these larger bodies of water, the plastic or polymeric microbeads may be ingested by wildlife or cause other environmental concerns. Thus, the possibility of producing microbead particles from more environmentally friendly materials has recently been explored. However, consumers tend to have high expectations when it comes to the personal care and/or cosmetic products they use, including those containing microbeads. For example, consumers expect these personal care and cosmetic products to exhibit certain optical properties that allow the products to blend in with natural skin color and readily hide any undesirable blemishes.

Thus, it is desirable to develop biodegradable microbeads that meet environmental and consumer expectation standards, particularly regarding optical properties for personal care and cosmetic products.

### SUMMARY

In one aspect, the present technology concerns biodegradable microparticles comprising a mixed cellulose ester, wherein the biodegradable microparticles exhibit at least 50 percent biodegradability at 60 days according to at least one of the OECD 301B, OECD 301C, or OECD 301 F test methods and a haze transmission of at least 10 percent. Furthermore, the mixed cellulose ester comprises: (a) an average degree of substitution for acetyl substituents (“DS_{Ac}”) in the range of 0.1 to 2.3, (b) an average degree of substitution for propionyl substituents (“DS_{Pr}”) or an average degree of substitution for butyryl substituents (“DS_{Bu}”) in the range of 0.1 to 1.5, and (c) an average degree of substitution for hydroxyl substituents (“DS_{OH}”) in the range of 0.6 to 2.8.

In one aspect, the present technology concerns biodegradable microparticles comprising a mixed cellulose ester, wherein the biodegradable microparticles exhibit at least 50 percent biodegradability at 60 days according to at least one of the OECD 301B, OECD 301C, or OECD 301F test methods and a haze transmission of at least 10 percent. Furthermore, the mixed cellulose ester comprises: (a) an average degree of substitution for acetyl substituents (“DS_{Ac}”) in the range of 0.1 to 2.3, (b) an average degree of substitution for butyryl substituents (“DS_{Bu}”) in the range of 0.1 to 1.5, and (c) an average degree of substitution for hydroxyl substituents (“DS_{OH}”) in the range of 0.6 to 2.8.

In one aspect, the present technology concerns a process for forming biodegradable microparticles. Generally, the process comprises: (a) forming a dope comprising a mixed cellulose ester; (b) contacting at least a portion of the dope with an aqueous mixture under agitation to thereby form an initial emulsion; (c) converting at least a portion of the initial emulsion into a dispersion comprising a solid phase and a liquid phase, wherein the solid phase comprises biodegradable microparticles; and (d) recovering at least a portion of the biodegradable microparticles from the dispersion. The biodegradable microparticles exhibit at least 50 percent biodegradability at 60 days according to at least one of the OECD 301B, OECD 301C, or OECD 301F test methods and a haze transmission of at least 10 percent. Furthermore, the mixed cellulose ester comprises: (a) an average degree of substitution for acetyl substituents (“DS_{Ac}”) in the range of 0.1 to 2.3, (b) an average degree of substitution for propionyl substituents (“DS_{Pr}”) or an average degree of substitution for butyryl substituents (“DS_{Bu}”) in the range of 0.1 to 1.5, and (c) an average degree of substitution for hydroxyl substituents (“DS_{OH}”) in the range of 0.6 to 2.8.

### BRIEF DESCRIPTION OF THE FIGURES

FIG. 1 is a schematic diagram of an example process for making cellulose ester microbeads.

FIG. 2 is a more detailed schematic diagram of an example process for making cellulose ester microbeads

FIG. 3 is a schematic diagram of an alternative process for making cellulose ester microbeads by serialized particle formation.

FIG. 4 is a schematic diagram of an alternative process for making cellulose ester microbeads by separated dope and aqueous mixture formation, and combined emulsion/dispersion formation and particle hardening.

FIG. 5 is a schematic diagram of an alternative process for making cellulose ester microbeads by combined emulsion/dispersion formation.

FIG. 6 is a schematic diagram of an alternative process for making cellulose ester microbeads by combined emulsion/dispersion formation and particle hardening.

FIG. 7 is a schematic diagram of an alternative process for making cellulose ester microbeads by solvent flashing prior to particle hardening.

### DETAILED DESCRIPTION

The present disclosure is directed to compositions and processes for making hardened or jet milled cellulose ester (CE) microbeads that exhibit superior optical properties. The processes disclosed herein enable the solidity of the produced microbeads to be controlled such that they exhibit desirable tactile and optical qualities. For example, the CE microbeads may have enhanced solidity, sphericity, and smoothness, low moisture content, and/or low free acid content. These qualities, along with the biodegradability of CE, make the CE microbeads produced herein desirable for use in personal care products, cosmetics, and the like.

The present invention may be understood more readily by reference to the following detailed description and the examples provided therein. It is to be understood that this disclosure is not limited to the specific methods, formulations, and conditions described, as such may vary. It is also to be understood that the terminology used herein is for the purpose of describing particular aspects of the disclosed embodiments only and is not intended to be limiting.

Values may be expressed as “about” or “approximately” a given number. Similarly, ranges may be expressed herein as from “about” one particular value and/or to “about” or another particular value. When such a range is expressed, another aspect includes from the one particular value and/or to the other particular value. Similarly, when values are expressed as approximations, by use of the antecedent “about,” it will be understood that the particular value forms another aspect.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “includes,” and “include” have the same open-ended meaning as “comprising,” “comprises,” and “comprise” provided above.

As used herein, a “mixed cellulose ester” shall denote a cellulose ester having at least two different ester substituents on a single cellulose ester polymer chain.

“Degree of Substitution” is used to describe the average substitution level of the substituents per anhydroglucose unit (“AGU”). Generally, conventional cellulose contains three hydroxyl groups in each AGU that can be substituted. Therefore, the DS can have a value between 0 and 3. However, low molecular weight cellulose mixed esters can have a total degree of substitution slightly above 3 from end group contributions. Low molecular weight cellulose mixed esters are discussed in more detail subsequently in this disclosure. Because DS is a statistical mean value, a value of 1 does not assure that every AGU has a single substituent. In some cases, there can be unsubstituted anhydroglucose units, some with two and some with three substituents, and more often than not the value will be a noninteger. Total DS is defined as the average number of all of substituents per anhydroglucose unit. The degree of substitution per AGU can also refer to a particular substituent, such as, for example, hydroxyl, acetyl, butyryl, or propionyl. Additionally, the degree of substitution can specify a given hydroxyl based on the carbon unit of the anhydroglucose unit.

“Spheroid” in reference to shape means that the average sphericity of the particles is less than 70%. “Spherical” in reference to shape means that the average sphericity of the particles is equal to or greater than 70%.

When the degree of substitution refers to hydroxyl, i.e, DS_{OH}, the reference is to the average hydroxyl groups per anhydroglucose that are not substituted. As a result, DS_{OH} is not used in the calculation of the total degree of substitution.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. For example, if the specification describes a specific temperature of 62° F., such a description provides literal support for a broad numerical range of 25° F. to 99° F. (62° F.+/−37° F.), an intermediate numerical range of 43° F. to 81° F. (62 □F+/−19° F.), and a narrow numerical range of 53° F. to 71° F. (62° F.+/−9° F.). These broad, intermediate, and narrow numerical ranges should be applied not only to the specific values, but should also be applied to differences between these specific values. Thus, if the specification describes a first pressure of 110 psia and a second pressure of 48 psia (a difference of 62 psi), the broad, intermediate, and narrow ranges for the pressure difference between these two streams would be 25 to 99 psi, 43 to 81 psi, and 53 to 71 psi, respectively.

Throughout this application, where patents or publications are referenced, the disclosures of these references in their entireties are intended to be incorporated by reference into this application, to the extent they are not inconsistent with the present invention, in order to more fully describe the state of the art to which the invention pertains.

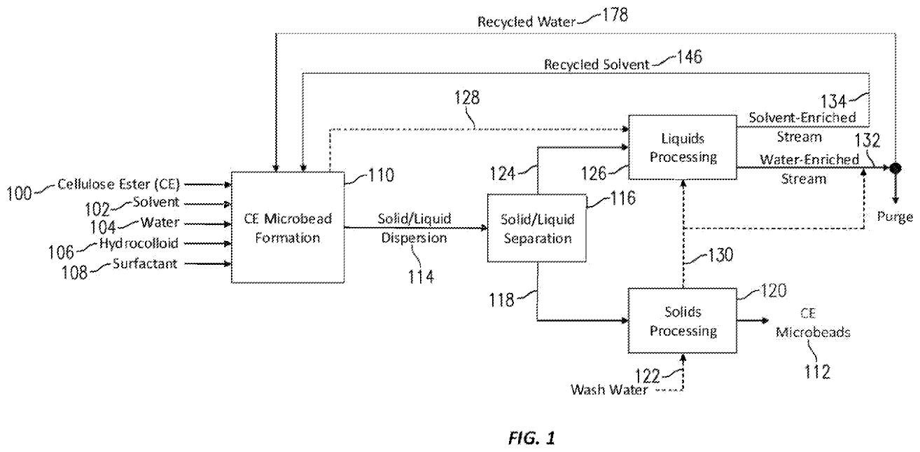

Referring now to FIG. 1, cellulose ester (CE) 100, solvent 102, water 104, hydrocolloid 106, and surfactant 108 may be combined in one or more units to make CE microbeads 112. In the illustrated example, CE 100, solvent 102, water 104, hydrocolloid 106, and surfactant 108 are combined at unit 110 to form an initial emulsion therein. The initial emulsion includes a dispersed phase and a continuous phase. The dispersed phase includes at least a portion of CE 100 and at least a portion of solvent 102. The continuous phase includes at least a portion of water 104, at least a portion of hydrocolloid 106, and at least a portion of surfactant 108.

Once combined, the initial emulsion may be agitated at unit 110 to form a pre-hardened dispersion including a sold phase and a liquid phase. The solid phase includes initial microparticles including at least a portion of CE 100 and at least a portion of solvent 102. The liquid phase includes at least a portion of water 104, at least a portion of hydrocolloid 106, and at least a portion of surfactant 108. Additional water 104 (i.e., a drowning liquid/extractant) may be added to the pre-hardened dispersion to increase the concentration of water around the initial microparticles. An initial hardening dispersion including the initial microparticles and the drowning liquid is formed therefrom. The initial hardening dispersion may be agitated at unit 110 to promote the transfer of solvent from the initial microparticles to the drowning liquid. This solvent transfer facilitates hardening the initial microparticles to produce hardened CE microbeads 112, which are entrained in a solvent-ladened drowning liquid.

CE microbeads 112 may be recovered from this dispersion 114 first by processing at a solid/liquid separation unit 116. A solid stream 118 including CE microbeads 112 may be channeled to a solids processing unit 120, where CE microbeads 112 are washed with water 122 and then dried to recover CE microbeads 112, as will be described in more detail below. A liquid stream 124 formed from the solvent-ladened drowning liquid may be channeled to a liquids processing unit 126. Optionally, an excess liquid stream 128 channeled from unit 110 and a wash water stream 130 from unit 120 may also be received at unit 126 for processing therein.

At unit 126, the liquids received therein may be separated into at least a water-enriched stream 132 and a solvent-enriched stream 134. In some embodiments, water-enriched stream 132 may be recovered and at least a portion of which utilized in unit 110, for example. In addition, solvent-enriched stream 134 may be recycled and utilized as at least a portion of the solvent for forming CE microbeads 112 in unit 110. Re-utilization of the recovered water and/or solvent facilitates improving the economics of the CE microbead formation processes described herein.

### Mixed Cellulose Esters

In one embodiment or in combination with any embodiment mentioned herein, the CE 100 can be a mixed cellulose ester.

Generally, the cellulose esters described herein, such as CE 100, can be produced by any method known in the art. Examples of processes for producing cellulose esters are taught in Kirk-Othmer, Encyclopedia of Chemical Technology, 5th Edition, Vol. 5, Wiley-Interscience, New York (2004), pp. 394-444, the disclosure of which is incorporated by reference in its entirety. Cellulose, the starting material for producing cellulose esters, can be obtained in different grades and from sources such as, for example, lignocellulosic sources (e.g., softwood pulp, hardwood pulp), cotton linters, corn fiber and other agricultural sources, and bacterial celluloses.

One method of producing cellulose esters is by esterification. In such a method, the cellulose is mixed with the appropriate organic acids, acid anhydrides, and/or catalysts and then converted to a cellulose triester. Ester hydrolysis is then performed by adding a water-acid mixture to the cellulose triester, which can be filtered to remove any gel particles or fibers. Water is added to the mixture to precipitate out the cellulose ester. The cellulose ester can then be washed with water to remove reaction by-products followed by dewatering and drying.

Acylating reagents suitable for use herein can include, but are not limited to, alkyl or aryl carboxylic anhydrides, carboxylic acid halides, and/or carboxylic acid esters containing the above-described alkyl or aryl groups suitable for use in the acyl substituents of the substituted cellulose esters described herein. Examples of suitable carboxylic anhydrides include, but are not limited to, acetic anhydride, propionic anhydride, butyric anhydride, pivaloyl anhydride, benzoic anhydride, and naphthoyl anhydride. Examples of carboxylic acid halides include, but are not limited to, acetyl, propionyl, butyryl, pivaloyl, benzoyl, and naphthoyl chlorides or bromides. Examples of carboxylic acid esters include, but are not limited to, acetyl, propionyl, butyryl, pivaloyl, benzoyl and naphthoyl methyl esters. In one or more embodiments, the acylating reagent can be one or more carboxylic anhydrides selected from the group consisting of acetic anhydride, propionic anhydride, butyric anhydride, pivaloyl anhydride, benzoyl anhydride, and naphthoyl anhydride.

In various embodiments, the cellulose triesters that are hydrolyzed can have three substituents selected independently from alkanoyls having from 2 to 12 carbon atoms. Examples of cellulose triesters include cellulose triacetate, cellulose tripropionate, cellulose tributyrate, or mixed triesters of cellulose, such as cellulose acetate propionate and cellulose acetate butyrate. These cellulose triesters can be prepared by a number of methods known to those skilled in the art. For example, cellulose triesters can be prepared by heterogeneous acylation of cellulose in a mixture of carboxylic acid and anhydride in the presence of a catalyst, such as H₂SO₄. Cellulose triesters can also be prepared by the homogeneous acylation of cellulose dissolved in an appropriate solvent such as LiCl/DMAc or LiCl/NMP.

After esterification of the cellulose to the triester, part of the acyl substituents can be removed by hydrolysis or by alcoholysis to give a secondary cellulose ester. Secondary cellulose esters can also be prepared directly with no hydrolysis by using a limiting amount of acylating reagent. This process is particularly useful when the reaction is conducted in a solvent that will dissolve cellulose.

The cellulose esters thus prepared generally comprise the following structure:

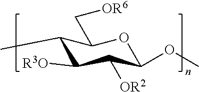

where R², R³, and R⁶ are hydrogen (with the proviso that R², R³, and R⁶ are not hydrogen simultaneously), alkyl-acyl groups, and/or aryl-acyl groups (such as those described above) bound to the cellulose via an ester linkage.

The degree of polymerization (“DP”) of the cellulose esters prepared by these methods can be at least 10. In other embodiments, the DP of the cellulose esters can be at least 50, at least 100, or at least 250. In other embodiments, the DP of the cellulose esters can be in the range of from about 5 to about 100, or in the range of from about 10 to about 50.

Acylating reagents suitable for use herein can include, but are not limited to, alkyl or aryl carboxylic anhydrides, carboxylic acid halides, and/or carboxylic acid esters containing the above-described alkyl or aryl groups suitable for use in the acyl substituents of the substituted cellulose esters described herein. Examples of suitable carboxylic anhydrides include, but are not limited to, acetic anhydride, propionic anhydride, butyric anhydride, pivaloyl anhydride, benzoic anhydride, and naphthoyl anhydride. Examples of carboxylic acid halides include, but are not limited to, acetyl, propionyl, butyryl, pivaloyl, benzoyl, and naphthoyl chlorides or bromides. Examples of carboxylic acid esters include, but are not limited to, acetyl, propionyl, butyryl, pivaloyl, benzoyl and naphthoyl methyl esters. In one or more embodiments, the acylating reagent can be one or more carboxylic anhydrides selected from the group consisting of acetic anhydride, propionic anhydride, butyric anhydride, pivaloyl anhydride, benzoyl anhydride, and naphthoyl anhydride.

The present application discloses, in a first aspect, a mixed ester cellulose ester (“MCE”), comprising: (1) a plurality of acetyl substituents; (2) a plurality of propionyl substituents; and (3) a plurality of hydroxyl substituents, wherein: the MCE has an average degree of substitution for the acetyl substituents (“DS_{Ac}”) is from 0.1 to 2.3, the MCE has an average degree of substitution for the propionyl substituents (“DS_{Pr}”) is from 0.1 to 1.5, the MCE has an average degree of substitution for the hydroxyl substituents (“DS_{OH}”) is from 0.6 to 2.8.

In one embodiment or in combination with any other embodiment, class or subclass of this first aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0, or from 1.8 to 2.3, or from 1.8 to 2.2, or from 1.8 to 2.1, or from 1.8 to 2.0, or from 1.85 to 2.3, or from 1.85 to 2.2, or from 1.85 to 2.1, or from 1.85 to 2.0, or from 1.9 to 2.3, or from 1.9 to 2.2, or from 1.9 to 2.1, or from 1.9 to 2.0. Additionally, or in the alternative, the DS_{Ac} is less than 2.3, or less than 2.2, or less than 2.1, or less than 2.0, or less than 1.9, or less than 1.8, or less than 1.7, or less than 1.6, or less than 1.5, or less than 1.4, or less than 1.3, or less than 1.2, or less than 1.1, or less than 1.0, or less than 0.9, or less than 0.8, or less than 0.7, or less than 0.5, or less than 0.4, or less than 0.3.

In one embodiment or in combination with any other embodiment, class or subclass of this first aspect, wherein the DS_{Ac} is from 0.6 to 2.2, or 0.6 to 2.1, or 0.6 to 2.0, or 0.6 to 1.9, or 0.6 to 1.8, or 0.7 to 2.3, or 0.7 to 2.2, or 0.7 to 2.1, or 0.7 to 2.0, or 0.7 to 1.9, or 0.8 to 2.3, or 0.8 to 2.2, or 0.8 to 2.1, or 0.8 to 2.0, or 0.8 to 1.9, or 0.9 to 2.3, or 0.9 to 2.2, or 0.9 to 2.1, or 0.9 to 2.0, or 0.9 to 1.9, or 1.0 to 2.3 or 1.0 to 2.2, or 1.0 to 2.1, or 1.0 to 2.0, or 1.0 to 1.9, or 1.1 to 2.3, or 1.1 to 2.2, or 1.1 to 2.1, or 1.1 to 2.0, or 1.1 to 1.9, or 1.2 to 2.3 or 1.2 to 2.2, or 1.2 to 2.1, or 1.2 to 2.0, or 1.2 to 1.9, or 0.6 to 1.5, or 0.6 to 1.3, or 0.6 to 1.1, or 0.6 to 0.9 or 0.7 to 1.5, or 0.7 to 1.3, or 0.7 to 1.1, or 0.7 to 0.9.

In one embodiment or in combination with any other embodiment, class or subclass of this first aspect, wherein the DS_{Pr} is at least 0.1, or at least 0.2, or at least 0.3, or at least 0.4, or at least 0.5, or at least 0.6, or at least 0.7, or at least 0.8, or at least 0.9, or at least 1.0, or at least 1.1, or at least 1.2, or at least 1.3, or at least 1.4. Additionally, or in the alternative, the DS_{Pr} is less than 1.5, or less than 1.4, or less than 1.3, or less than 1.2, or less than 1.1, or less than 1.0, or less than 0.9, or less than 0.8, or less than 0.7, or less than 0.5, or less than 0.4, or less than 0.3.

In one embodiment or in combination with any other embodiment, class or subclass of this first aspect, wherein the DS_{Pr} is from 0.1 to 0.9, or 0.1 to 0.85, or 0.1 to 0.8, or 0.1 to 0.75, or 0.1 to 0.7, or 0.1 to 0.6 or 0.1 to 0.5, or 0.1 to 0.4, or 0.15 to 0.95, or 0.15 to 0.9, or 0.15 to 0.85, or 0.15 to 0.8, or 0.15 to 0.75, or 0.15 to 0.7, or 0.15 to 0.65, or 0.2 to 0.95, or 0.2 to 0.9, or 0.2 to 0.85, or 0.2 to 0.8, or 0.2 to 0.75, or 0.2 to 0.7, or 0.2 to 0.65, 0.25 to 0.95, or 0.25 to 0.9, or 0.25 to 0.85, or 0.25 to 0.8, or 0.25 to 0.75, or 0.25 to 0.7, or 0.25 to 0.65, or 0.3 to 0.95, or 0.3 to 0.9, or 0.3 to 0.85, or 0.3 to 0.8, or 0.3 to 0.75, or 0.3 to 0.7, or 0.3 to 0.65, or 0.35 to 0.95, or 0.35 to 0.9, or 0.35 to 0.85, or 0.35 to 0.8, or 0.35 to 0.75, or 0.35 to 0.7, or 0.35 to 0.65, or 0.4 to 0.95, or 0.4 to 0.9, or 0.4 to 0.85, or 0.4 to 0.8, or 0.4 to 0.75, or 0.4 to 0.7, or 0.4 to 0.65, or 0.45 to 0.95, or 0.45 to 0.9, or 0.45 to 0.85, or 0.45 to 0.8, or 0.45 to 0.75, or 0.45 to 0.7, or 0.45 to 0.65, or 0.5 to 0.95, or 0.5 to 0.9, or 0.5 to 0.85, or 0.5 to 0.8, or 0.5 to 0.75, or 0.5 to 0.7, or 0.5 to 0.65, or 0.1 to 0.9, or 0.1 to 0.85, or 0.1 to 0.8.

In one embodiment or in combination with any other embodiment, class or subclass of this first aspect, wherein the DS_{OH} is at least 0.6, or at least 0.7, or at least 0.8, or at least 0.9, or at least 1.0, or at least 1.1, or at least 1.2, or at least 1.3, or at least 1.4, or at least 1.5, or at least 1.6, or at least 1.7, or at least 1.8, or at least 1.9, or at least 2.0, or at least 2.1, or at least 2.2, or at least 2.3, or at least 2.4, or at least 2.5, or at least 2.6. Additionally, or in the alternative, the DS_{OH} is less than 2.8, or less than 2.7, or less than 2.6, or less than 2.5, or less than 2.4, or less than 2.3, or less than 2.2, or less than 2.1, or less than 2.0, or less than 1.9, or less than 1.8, or less than 1.7, or less than 1.6, or less than 1.5, or less than 1.4, or less than 1.3, or less than 1.2, or less than 1.1, or less than 1.0, or less than 0.9, or less than 0.8.

In one embodiment or in combination with any other embodiment, class or subclass of this first aspect, wherein the DS_{OH} is from 0.5 to 1.5, or 0.5 to 1.45, or 0.5 to 1.40, or 0.5 to 1.35, or 0.5 to 1.30, or 0.5 to 1.25, or 0.5 to 1.2, or 0.5 to 1.15, or 0.5 to 1.1, or 0.5 to 1.05, or 0.5 to 1.0, or 0.5 to 0.95 or 0.5 to 0.9, or 0.55 to 1.5, or 0.55 to 1.45, or 0.55 to 1.40, or 0.55 to 1.35, or 0.55 to 1.30, or 0.55 to 1.25, or 0.55 to 1.2, or 0.55 to 1.15, or 0.55 to 1.1, or 0.55 to 1.05, or 0.55 to 1.0, or 0.55 to 0.95 or 0.55 to 0.9, or 0.6 to 1.5, or 0.6 to 1.45, or 0.6 to 1.40, or 0.6 to 1.35, or 0.6 to 1.30, or 0.6 to 1.25, or 0.6 to 1.2, or 0.6 to 1.15, or 0.6 to 1.1, or 0.6 to 1.05, or 0.6 to 1.0, or 0.6 to 0.95 or 0.6 to 0.9, or 0.65 to 1.5, or 0.65 to 1.45, or 0.65 to 1.40, or 0.65 to 1.35, or 0.65 to 1.30, or 0.65 to 1.25, or 0.65 to 1.2, or 0.65 to 1.15, or 0.65 to 1.1, or 0.65 to 1.05, or 0.65 to 1.0, or 0.65 to 0.95 or 0.65 to 0.9, or 0.7 to 1.5, or 0.7 to 1.45, or 0.7 to 1.40, or 0.7 to 1.35, or 0.7 to 1.30, or 0.7 to 1.25, or 0.7 to 1.2, or 0.7 to 1.15, or 0.7 to 1.1, or 0.7 to 1.05, or 0.7 to 1.0, or 0.7 to 0.95 or 0.7 to 0.9.

In one embodiment or in combination with any other embodiment, class or subclass of this first aspect, wherein the sum of DS_{Pr} and DS_{Ac} is from 1.9 to 2.44, or 1.9 to 2.0, or 1.9 to 2.1, or 1.9 to 2.2, or 1.9 to 2.3, or 2.0 to 2.44, or 2.0 to 2.1, or 2.0 to 2.2, or 2.0 to 2.3, or 2.1 to 2.44, or 2.1 to 2.2, or 2.1 to 2.3, or 2.2 to 2.44, or 2.2 to 2.3.

In one embodiment or in combination with any other embodiment, class or subclass of this first aspect, wherein the mixed cellulose ester has a ratio of hydroxyl substituents to acetyl substituents of at least 0.4:1, or at least 0.5:1, or at least 0.6:1, or at least 0.7:1, or at least 0.8:1, or at least 0.9:1, or at least 1:1, or at least 1.1:1, or at least 1.2:1, or at least 1.3:1, or at least 1.4:1, or at least 1.5:1, or at least 1.6:1, or at least 1.7:1, or at least 1.8:1, or at least 1.9:2, or at least 2:1. Additionally, in the alternative, the mixed cellulose ester has a ratio of hydroxyl substituents to acetyl substituents of less than 2:1, or less than 1.9:1, or less than 1.8:1, or less than 1.7:1, or less than 1.6:1, or less than 1.5:1, or less than 1.4:1, or less than 1.3:1, or less than 1.2:1, or less than 1.1:1, or less than 1:1.

In one embodiment or in combination with any other embodiment, class or subclass of this first aspect, wherein the MCE has a ratio of hydroxyl substituents to propionyl substituents of at least 0.4:1, or at least 0.5:1, or at least 0.6:1, or at least 0.7:1, or at least 0.8:1, or at least 0.9:1, or at least 1:1, or at least 1.1:1, or at least 1.2:1, or at least 1.3:1, or at least 1.4:1, or at least 1.5:1, or at least 1.6:1, or at least 1.7:1, or at least 1.8:1, or at least 1.9:2, or at least 2:1. Additionally, or in the alternative, the MCE has a ratio of hydroxyl substituents to propionyl substituents of less than 2:1, or less than 1.9:1, or less than 1.8:1, or less than 1.7:1, or less than 1.6:1, or less than 1.5:1, or less than 1.4:1, or less than 1.3:1, or less than 1.2:1, or less than 1.1:1, or less than 1:1.

In one embodiment or in combination with any other embodiment, class or subclass of this first aspect, wherein the MCE exhibits at least 40% biodegradability, at least 45% biodegradability, or at least 50% biodegradability, or at least 55% biodegradability, or at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability, at 56 days according to at least one of the OECD 301B, OECD 301C, or OECD 301F test methods.

In one embodiment or in combination with any other embodiment, class or subclass of this first aspect, wherein the MCE exhibits at least 40% biodegradability, or at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability or at least 90% biodegradability, or at least 95% biodegradability at 60 days according to at least one of the OECD 301B, OECD 301C, or OECD 301F test methods.

In one embodiment or in combination with any other embodiment, class or subclass of this first aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

The present application discloses, in a second aspect, a mixed cellulose ester (“MCE”), comprising: (1) a plurality of acetyl substituents; (2) a plurality of propionyl substituents; and (3) a plurality of hydroxyl substituents, wherein: the MCE has an average degree of substitution for the acetyl substituents (“DS_{Ac}”) is from 0.1 to 1.9, the MCE has an average degree of substitution for the propionyl substituents (“DS_{Pr}”) is from 0.1 to 1.5, and the MCE has an average degree of substitution for the hydroxyl substituents (“DS_{OH}”) is from 0.7 to 2.8.

In one embodiment or in combination with any other embodiment, class or subclass of this second aspect, wherein the DS_{Ac} is at least 0.1, or at least 0.2, or at least 0.3, or at least 0.4, or at least 0.5, or at least 0.6, or at least 0.7, or at least 0.8, or at least 0.9, or at least 1.0, or at least 1.1, or at least 1.2, or at least 1.3, or at least 1.4, or at least 1.5, or at least 1.6, or at least 1.7, or at least 1.8, or at least 1.9, or at least 2.0. Additionally, or in the alternative, the DS_{Ac} is less than 2.3, or less than 2.2, or less than 2.1, or less than 2.0, or less than 1.9, or less than 1.8, or less than 1.7, or less than 1.6, less than 1.5, or less than 1.4, or less than 1.3, or less than 1.2, or less than 1.1, or less than 1.0, or less than 0.9, or less than 0.8, or less than 0.7, or less than 0.5, or less than 0.4, or less than 0.3.

In one embodiment or in combination with any other embodiment, class or subclass of this second aspect, wherein the DS_{Ac} is from 0.6 to 0.7, or 0.6 to 0.8, or 0.6 to 0.9, or 0.6 to 1.0, or 0.6 to 1.1, or 0.7 to 0.9, or 0.7 to 1.0, or 0.7 to 1.1, or 0.7 to 1.2, or 0.8 to 0.9, or 0.8 to 1.0, or 0.8 to 1.1, or 0.8 to 1.2, or 0.9 to 1.0, or 0.9 to 1.1, or 0.9 to 1.2, or 1.0 to 1.1, or 1.0 to 1.2, or 1.1 to 1.2.

In one embodiment or in combination with any other embodiment, class or subclass of this second aspect, wherein the DS_{Pr} is at least 0.1, or at least 0.2, or at least 0.3, or at least 0.4, or at least 0.5, or at least 0.6, or at least 0.7, or at least 0.8, or at least 0.9, or at least 1.0, or at least 1.1, or at least 1.2, or at least 1.3, or at least 1.4. Additionally, or in the alternative, the DS_{Pr} is less than 1.5, or less than 1.4, or less than 1.3, or less than 1.2, or less than 1.1, or less than 1.0, or less than 0.9, or less than 0.8, or less than 0.7, or less than 0.5, or less than 0.4, or less than 0.3.

In one embodiment or in combination with any other embodiment, class or subclass of this second aspect, wherein the DS_{Pr} is from 1.05 to 1.35, or 1.05 to 1.3, or 1.05 to 1.25, or 1.05 to 1.2, or 1.05 to 1.15, or 1.05 to 1.1, or 1.1 to 1.4, or 1.1 to 1.35, or 1.1 to 1.3, or 1.1 to 1.25, or 1.1 to 1.2, or 1.1 to 1.15, or 1.15 to 1.4, or 1.15 to 1.35, or 1.15 to 1.3, or 1.15 to 1.25, or 1.15 to 1.2, or 1.2 to 1.4, or 1.2 to 1.35, or 1.2 to 1.3, or 1.2 to 1.25, or 1.25 to 1.4, or 1.25 to 1.35, or 1.25 to 1.3, or 1.3 to 1.4, or 1.3 to 1.35.

In one embodiment or in combination with any other embodiment, class or subclass of this second aspect, wherein the DS_{OH} is at least 0.6, or at least 0.7, or at least 0.8, or at least 0.9, or at least 1.0, or at least 1.1, or at least 1.2, or at least 1.3, or at least 1.4, or at least 1.5, or at least 1.6, or at least 1.7, or at least 1.8, or at least 1.9, or at least 2.0, or at least 2.1, or at least 2.2, or at least 2.3, or at least 2.4, or at least 2.5, or at least 2.6. Additionally, or in the alternative, the DS_{OH} is less than 2.8, or less than 2.7, or less than 2.6, or less than 2.5, or less than 2.4, or less than 2.3, or less than 2.2, or less than 2.1, or less than 2.0, or less than 1.9, or less than 1.8, or less than 1.7, or less than 1.6, or less than 1.5, or less than 1.4, or less than 1.3, or less than 1.2, or less than 1.1, or less than 1.0, or less than 0.9, or less than 0.8.

In one embodiment or in combination with any other embodiment, class or subclass of this second aspect, wherein the DS_{OH} is from 0.7 to 1.35, or 0.7 to 1.3, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.7 to 0.75, or 0.75 to 1.4, or 0.75 to 1.35, or 0.75 to 1.3, or 0.75 to 1.25, or 0.75 to 1.2, or 0.75 to 1.15, or 0.75 to 1.1, or 0.75 to 1.05, or 0.75 to 1.0, or 0.75 to 0.95, or 0.8 to 1.4, or 0.8 to 1.35, or 0.8 to 1.3, or 0.8 to 1.25, or 0.8 to 1.2, or 0.8 to 1.15, or 0.8 to 1.1, or 0.8 to 1.05, or 0.85 to 1.4, or 0.85 to 1.35, or 0.85 to 1.3, or 0.85 to 1.25, or 0.85 to 1.2, or 0.85 to 1.15, or 0.85 to 1.1, or 0.85 to 1.05, or 0.9 to 1.4, or 0.9 to 1.35, or 0.9 to 1.3, or 0.9 to 1.25, or 0.9 to 1.2, or 0.9 to 1.15, or 0.9 to 1.1, or 0.9 to 1.05.

In one embodiment or in combination with any other embodiment, class or subclass of this second aspect, wherein the sum of DS_{Pr} and DS_{Ac} is from 1.65 to 2.3, or 1.65 to 2.2, or 1.65 to 2.1, or 1.65 to 2.0, or 1.65 to 1.9, or 1.65 to 1.8, or 1.7 to 2.3, or 1.7 to 2.2, or 1.7 to 2.1, or 1.7 to 2.0, or 1.7 to 1.9, or 1.7 to 1.8, or 1.75 to 2.3, or 1.75 to 2.2, or 1.75 to 2.1, or 1.75 to 2.0, or 1.75 to 1.9, or 1.8 to 2.3, or 1.8 to 2.2, or 1.8 to 2.1, or 1.8 to 2.0, or 1.8 to 1.9, or 1.9 to 2.3, or 1.9 to 2.2, or 1.9 to 2.1, or 1.9 to 2.0, or 2.0 to 2.3, or 2.0 to 2.2, or 2.0 to 2.1.

In one embodiment or in combination with any other embodiment, class or subclass of this second aspect, wherein the DS_{OH} is from 0.6 to 0.7, or 0.7 to 1.35, or 0.7 to 1.3, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.7 to 0.75, or 0.75 to 1.4, or 0.75 to 1.35, or 0.75 to 1.3, or 0.75 to 1.25, or 0.75 to 1.2, or 0.75 to 1.15, or 0.75 to 1.1, or 0.75 to 1.05, or 0.75 to 1.0, or 0.75 to 0.95, or 0.8 to 1.4, or 0.8 to 1.35, or 0.8 to 1.3, or 0.8 to 1.25, or 0.8 to 1.2, or 0.8 to 1.15, or 0.8 to 1.1, or 0.8 to 1.05, or 0.85 to 1.4, or 0.85 to 1.35, or 0.85 to 1.3, or 0.85 to 1.25, or 0.85 to 1.2, or 0.85 to 1.15, or 0.85 to 1.1, or 0.85 to 1.05, or 0.9 to 1.4, or 0.9 to 1.35, or 0.9 to 1.3, or 0.9 to 1.25, or 0.9 to 1.2, or 0.9 to 1.15, or 0.9 to 1.1, or 0.9 to 1.05.

In one embodiment or in combination with any other embodiment, class or subclass of this second aspect, wherein the mixed cellulose ester has a ratio of hydroxyl substituents to acetyl substituents of at least 0.4:1, or at least 0.5:1, or at least 0.6:1, or at least 0.7:1, or at least 0.8:1, or at least 0.9:1, or at least 1:1, or at least 1.1:1, or at least 1.2:1, or at least 1.3:1, or at least 1.4:1, or at least 1.5:1, or at least 1.6:1, or at least 1.7:1, or at least 1.8:1, or at least 1.9:2, or at least 2:1. Additionally, in the alternative, the mixed cellulose ester has a ratio of hydroxyl substituents to acetyl substituents of less than 2:1, or less than 1.9:1, or less than 1.8:1, or less than 1.7:1, or less than 1.6:1, or less than 1.5:1, or less than 1.4:1, or less than 1.3:1, or less than 1.2:1, or less than 1.1:1, or less than 1:1.

In one embodiment or in combination with any other embodiment, class or subclass of this second aspect, wherein the MCE has a ratio of hydroxyl substituents to propionyl substituents of at least 0.4:1, or at least 0.5:1, or at least 0.6:1, or at least 0.7:1, or at least 0.8:1, or at least 0.9:1, or at least 1:1, or at least 1.1:1, or at least 1.2:1, or at least 1.3:1, or at least 1.4:1, or at least 1.5:1, or at least 1.6:1, or at least 1.7:1, or at least 1.8:1, or at least 1.9:2, or or at least 2:1. Additionally, or in the alternative, the MCE has a ratio of hydroxyl substituents to propionyl substituents of less than 2:1, or less than 1.9:1, or less than 1.8:1, or less than 1.7:1, or less than 1.6:1, or less than 1.5:1, or less than 1.4:1, or less than 1.3:1, or less than 1.2:1, or less than 1.1:1, or less than 1:1.

In one embodiment or in combination with any other embodiment, class or subclass of this second aspect, wherein the MCE exhibits at least 40% biodegradability, at least 45% biodegradability, or at least 50% biodegradability, or at least 55% biodegradability, or at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability at 56 days according to at least one of the OECD 301B, OECD 301C, or OECD 301F test methods.

In one embodiment or in combination with any other embodiment, class or subclass of this second aspect, wherein the MCE exhibits at least 40% biodegradability, at least 45% biodegradability, or at least 50% biodegradability, or at least 55% biodegradability, or at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability at 60 days according to at least one of the OECD 301B, OECD 301C, or OECD 301F test methods.

In one embodiment or in combination with any other embodiment, class or subclass of this second aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

The present application, in a third aspect, also discloses a mixed cellulose ester (“MCE”), comprising: (1) a plurality of acetyl substituents; (2) a plurality of butyryl substituents; and (3) a plurality of hydroxyl substituents, wherein: the MCE has an average degree of substitution for the acetyl substituents (“DS_{Ac}”) is from 0.1 to 2.4, the MCE has an average degree of substitution for the butyryl substituents (“DS_{B}u”) is from 0.1 to 1.5, the MCE has an average degree of substitution for the hydroxyl substituents (“DS_{OH}”) is from 0.6 to 2.8.

In one embodiment or in combination with any other embodiment, class or subclass of this first aspect, wherein the DS_{Ac} is at least 0.1, or at least 0.2, at least 0.3, or at least 0.4, or at least 0.5, or at least 0.6, or at least 0.7, or at least 0.8, or at least 0.9, or at least 1.0, or at least 1.1, or at least 1.2, or at least 1.3, or at least 1.4, or at least 1.5, or at least 1.6, or at least 1.7, or at least 1.8, or at least 1.9, or at least 2.0. Additionally, or in the alternative, the DS_{Ac} is less than 2.3, or less than 2.2, or less than 2.1, or less than 2.0, or less than 1.9, or less than 1.8, or less than 1.7, or less than 1.6, or less than 1.5, or less than 1.4, or less than 1.3, or less than 1.2, or less than 1.1, or less than 1.0, or less than 0.9, or less than 0.8, or less than 0.7, or less than 0.5, or less than 0.4, or less than 0.3.

In one embodiment or in combination with any other embodiment, class or subclass of this third aspect, wherein the DS_{Ac} is from 0.9 to 2.4, or 0.9 to 2.3, or 0.9 to 2.2, or 0.9 to 2.1, or 0.9 to 2.0, or 0.9 to 1.9, or 0.9 to 1.8, or 0.9 to 1.7, or 0.9 to 1.6, or 0.9 to 1.4, or 0.9 to 1.3, or 0.9 to 1.2, or 0.9 to 1.1, or 0.9 to 1.0, or 0.92 to 2.4, or 0.92 to 2.3, or 0.92 to 2.2, or 0.92 to 2.1, or 0.92 to 2.0, or 0.92 to 1.9, or 0.92 to 1.8, or 0.92 to 1.7, or 0.92 to 1.6, or 0.92 to 1.4, 0.92 to 1.3, or 0.92 to 1.2, or 0.92 to 1.1, or 0.92 to 1.0, or 0.94 to 2.4, 0.94 to 2.3, or 0.94 to 2.2, or 0.94 to 2.1, or 0.94 to 2.0, or 0.94 to 1.9, or 0.94 to 1.8, or 0.94 to 1.7, or 0.94 to 1.6, or 0.94 to 1.4, 0.94 to 1.3, or 0.94 to 1.2, or 0.94 to 1.1, or 0.94 to 1.0, or 0.96 to 2.4, or 0.96 to 2.3, or 0.96 to 2.2, or 0.96 to 2.1, or 0.96 to 2.0, or 0.96 to 1.9, or 0.96 to 1.8, or 0.96 to 1.7, or 0.96 to 1.6, or 0.96 to 1.4, 0.96 to 1.3, or 0.96 to 1.2, or 0.96 to 1.1, or 0.96 to 1.0, or 0.98 to 2.4, 0.98 to 2.3, or 0.98 to 2.2, or 0.98 to 2.1, or 0.98 to 2.0, or 0.98 to 1.9, or 0.98 to 1.8, or 0.98 to 1.7, or 0.98 to 1.6, or 0.98 to 1.4, 0.98 to 1.3, or 0.98 to 1.2, or 0.98 to 1.1, or 0.98 to 1.0, or 1.0 to 2.4, or 1.0 to 2.3, or 1.0 to 2.2, or 1.0 to 2.1, or 1.0 to 2.0, or 1.0 to 1.9, or 1.0 to 1.8, or 1.0 to 1.7, or 1.0 to 1.6, or 1.0 to 1.4, or 1.0 to 1.3, 1.0 to 1.2, or 1.0 to 1.1, or 1.1 to 2.4, or 1.1 to 2.3, or 1.1 to 2.2, or 1.1 to 2.1, or 1.1 to 2.0, or 1.1 to 1.9, or 1.1 to 1.8, or 1.1 to 1.7, or 1.1 to 1.6, or 1.1 to 1.4, or 1.1 to 1.3, or 1.1 to 1.2, or 1.2 to 2.4, or 1.2 to 2.3, or 1.2 to 2.2, or 1.2 to 2.1, or 1.2 to 2.0, or 1.2 to 1.9, or 1.2 to 1.8, or 1.2 to 1.7, or 1.2 to 1.6, or 1.2 to 1.4, or 1.2 to 1.3, or 1.3 to 2.4, or 1.3 to 2.3, or 1.3 to 2.2, or 1.3 to 2.1, or 1.3 to 2.0, or 1.3 to 1.9, or 1.3 to 1.8, or 1.3 to 1.7, or 1.3 to 1.6, or 1.3 to 1.4, or 1.4 to 2.4, or 1.4 to 2.3, or 1.4 to 2.2, or 1.4 to 2.1, or 1.4 to 2.0, or 1.4 to 1.9, or 1.4 to 1.8, or 1.4 to 1.7, or 1.4 to 1.6, or 1.5 to 2.4, or 1.5 to 2.3, or 1.5 to 2.2, or 1.5 to 2.1, or 1.5 to 2.0, or 1.5 to 1.9, or 1.5 to 1.8, or 1.5 to 1.7, or 1.5 to 1.6, or 1.6 to 2.4, or 1.6 to 2.3, or 1.6 to 2.2, or 1.6 to 2.1, or 1.6 to 2.0, or 1.6 to 1.9, or 1.6 to 1.8, or 1.6 to 1.7, or 1.7 to 2.4, or 1.7 to 2.3, or 1.7 to 2.2, or 1.7 to 2.1, or 1.7 to 2.0, or 1.7 to 1.9, or 1.7 to 1.8, or 1.8 to 2.3, or 1.8 to 2.1, or 1.8 to 2.0, or 1.8 to 1.9, or 1.9 to 2.3, or 1.9 to 2.2, or 1.9 to 2.1, or 1.9 to 2.0, or 2.0 to 2.4, or 2.0 to 2.3, or 2.0 to 2.2, or 2.0 to 2.1, or 2.1 to 2.4, or 2.1 to 2.3, or 2.1 to 2.2, or 2.2 to 2.3.

In one embodiment or in combination with any other embodiment, class or subclass of this third aspect, wherein the DS_{Bu} is at least 0.2, or at least 0.3, or at least 0.4, or at least 0.5, or at least 0.6, or at least 0.7, or at least 0.8, or at least 0.9, or at least 1.0, or at least 1.1, or at least 1.2, or at least 1.3, or at least 1.4, or from 0.18 to 0.23, or from 0.18 to 0.22, or from 0.18 to 0.21, or from 0.18 to 0.20, or from 0.18 to 0.19, or from 0.19 to 0.23, or from 0.19 to 0.22, or from 0.19 to 0.21, or from 0.19 to 0.2, or from 0.2 to 0.23, or from 0.2 to 0.22, or from 0.2 to 0.21. Additionally, or in the alternative, the DS_{Bu} is less than 1.5, or less than 1.4, or less than 1.3, or less than 1.2, or less than 1.1, or less than 1.0, or less than 0.9, or less than 0.8, or less than 0.7, or less than 0.5, or less than 0.4, or less than 0.3.

In one embodiment or in combination with any other embodiment, class or subclass of this third aspect, wherein the DS_{Bu} is from 0.1 to 1.35, or 0.1 to 1.3, or 0.1 to 1.25, or 0.1 to 1.2, or 0.1 to 1.15, or 0.1 to 1.1, or 0.1 to 1.0, or 0.1 to 0.8, or 0.1 to 0.6, or 0.2 to 1.35, or 0.2 to 1.3, or 0.2 to 1.25, or 0.2 to 1.2, or 0.2 to 1.15, or 0.2 to 1.1, or 0.2 to 1.0, or 0.2 to 0.8, or 0.2 to 0.6, or 0.2 to 0.4, or 0.3 to 1.35, or 0.3 to 1.3, or 0.3 to 1.25, or 0.3 to 1.2, or 0.3 to 1.15, or 0.3 to 1.1, or 0.3 to 1.0, or 0.3 to 0.8, or 0.3 to 0.6, or 0.3 to 0.5, or 0.4 to 1.35, or 0.4 to 1.3, or 0.4 to 1.25, or 0.4 to 1.2, or 0.4 to 1.15, or 0.4 to 1.1, or 0.4 to 1.0, or 0.4 to 0.8, or 0.4 to 0.6, or 0.5 to 1.35, or 0.5 to 1.3, or 0.5 to 1.25, or 0.5 to 1.2, or 0.5 to 1.15, or 0.5 to 1.1, or 0.5 to 1.0, or 0.5 to 0.8, or 0.5 to 0.7, or 0.6 to 1.35, or 0.6 to 1.3, or 0.6 to 1.25, or 0.6 to 1.2, or 0.6 to 1.15, or 0.6 to 1.1, or 0.6 to 1.0, or 0.6 to 0.8, or 0.7 to 1.35, or 0.7 to 1.3, or 0.7 to 1.25, or 0.7 to 1.2, or 0.7 to 1.15, or 0.7 to 1.1, or 0.7 to 1.0, or 0.8 to 1.35, or 0.8 to 1.3, or 0.8 to 1.25, or 0.8 to 1.2, or 0.8 to 1.15, or 0.8 to 1.1, or 0.8 to 1.0, or 0.9 to 1.35, or 0.9 to 1.3, or 0.9 to 1.25, or 0.9 to 1.2, or 0.9 to 1.15, or 0.9 to 1.1, or 1.0 to 1.35, or 1.0 to 1.3, or 1.0 to 1.25, or 1.0 to 1.2, or 1.0 to 1.15, or 1.0 to 1.1, or 1.05 to 1.35, or 1.05 to 1.3, or 1.05 to 1.25, or 1.05 to 1.2, or 1.05 to 1.15, or 1.05 to 1.1, or 1.1 to 1.4, or 1.1 to 1.35, or 1.1 to 1.3, or 1.1 to 1.25, or 1.1 to 1.2, or 1.1 to 1.15, or 1.15 to 1.4, or 1.15 to 1.35, or 1.15 to 1.3, or 1.15 to 1.25, or 1.15 to 1.2, or 1.2 to 1.4, or 1.2 to 1.35, or 1.2 to 1.3, or 1.2 to 1.25, or 1.25 to 1.4, or 1.25 to 1.35, or 1.25 to 1.3, or 1.3 to 1.4, or 1.3 to 1.35.

In one embodiment or in combination with any other embodiment, class or subclass of this third aspect, wherein the DS_{OH} is at least 0.6, or at least 0.7, or at least 0.8, or at least 0.9, or at least 1.0, or at least 1.1, or at least 1.2, or at least 1.3, or or at least 1.4, or from 0.5 to 0.95, or from 0.5 to 0.93, or from 0.5 to 0.91, or from 0.5 to 0.9, or from 0.5 to 0.85, or from 0.5 to 0.80, or from 0.5 to 0.75, or from 0.5 to 0.7 or from 0.55 to 0.95, or from 0.55 to 0.93, or from 0.55 to 0.91, or from 0.55 to 0.9, or from 0.55 to 0.85, or from 0.55 to 0.80, or from 0.55 to 0.75, or from 0.55 to 0.7, or from 0.6 to 0.95, or from 0.6 to 0.93, or from 0.6 to 0.91, or from 0.6 to 0.9, or from 0.6 to 0.85, or from 0.6 to 0.80, or from 0.6 to 0.75, or from 0.6 to 0.7, or from 0.65 to 0.95, or from 0.65 to 0.93, or from 0.65 to 0.91, or from 0.65 to 0.9, or from 0.65 to 0.85, or from 0.65 to 0.80, or from 0.65 to 0.75, or from 0.65 to 0.7, or from 0.7 to 0.95, or from 0.7 to 0.93, or from 0.7 to 0.91, or from 0.7 to 0.9, or from 0.7 to 0.85, or from 0.7 to 0.80, or from 0.7 to 0.75, or from 0.8 to 0.95, or from 0.8 to 0.93, or from 0.8 to 0.91, or from 0.8 to 0.9, or from 0.8 to 0.85, or from 0.85 to 0.95, or from 0.85 to 0.93, or from 0.85 to 0.91, or from 0.85 to 0.9, or from 0.9 to 0.95, or from 0.9 to 0.93, or from 0.9 to 0.91. Additionally, or in the alternative, the DS_{OH} is less than 2.8, less than 2.7, or less than 2.6, or less than 2.5, or less than 2.4, or less than 2.3, or less than 2.2, or less than 2.1, less than 2.0, or less than 1.9, or less than 1.8, or less than 1.7, or less than 1.6, or less than 1.5, or less than 1.4, or less than 1.3, or less than 1.2, or less than 1.1, or less than 1.0, or less than 0.9, or less than 0.8. In one embodiment or in combination with any other embodiment, class or subclass of this third aspect, wherein the DS_{OH} is from 0.5 to 1.0, or 0.5 to 0.95, or 0.5 to 0.9, or 0.5 to 0.85, or 0.5 to 0.8, or 0.5 to 0.75, or 0.5 to 0.7, or 0.5 to 0.65, or 0.5 to 0.6, or 0.5 to 0.55, or 0.55 to 1.0, or 0.55 to 0.95, or 0.55 to 0.9, or 0.55 to 0.85, or 0.55 to 0.8, or 0.55 to 0.75, or 0.55 to 0.7, or 0.55 or 0.65, or 0.55 to 0.6, or 0.6 to 0.65, or 0.6 to 0.7, or 0.6 to 0.75, or 0.6 to 0.8, or 0.6 to 0.85, or 0.6 to 0.9, or 0.6 to 0.95, or 0.6 to 1.0, or 0.65 to 0.7, or 0.65 to 0.75, or 0.65 to 0.8, or 0.65 to 0.85, or 0.65 to 0.9, or 0.65 to 0.95, or 0.65 to 1.0.

In one embodiment or in combination with any other embodiment, class or subclass of this third aspect, wherein the sum of DS_{Bu} and DS_{Ac} is from 1.65 to 2.3, or 1.65 to 2.2, or 1.65 to 2.1, or 1.65 to 2.0, or 1.65 to 1.9, or 1.65 to 1.8, or 1.7 to 2.3, or 1.7 to 2.2, or 1.7 to 2.1, or 1.7 to 2.0, or 1.7 to 1.9, or 1.7 to 1.8, or 1.75 to 2.3, or 1.75 to 2.2, or 1.75 to 2.1, or 1.75 to 2.0, or 1.75 to 1.9, or 1.8 to 2.3, or 1.8 to 2.2, or 1.8 to 2.1, or 1.8 to 2.0, or 1.8 to 1.9, or 1.9 to 2.3, or 1.9 to 2.2, or 1.9 to 2.1, or 1.9 to 2.0, 2.0 to 2.4, or 2.0 to 2.3, or 2.0 to 2.2, or 2.0 to 2.1.

In one embodiment or in combination with any other embodiment, class or subclass of this third aspect, wherein the mixed cellulose ester has a ratio of hydroxyl substituents to acetyl substituents of at least 0.4:1, or at least 0.5:1, or at least 0.6:1, or at least 0.7:1, or at least 0.8:1, or at least 0.9:1, or at least 1:1, or at least 1.1:1, or at least 1.2:1, or at least 1.3:1, or at least 1.4:1, or at least 1.5:1, or at least 1.6:1, or at least 1.7:1, or at least 1.8:1, or at least 1.9:2, or at least 2:1. Additionally, in the alternative, the mixed cellulose ester has a ratio of hydroxyl substituents to acetyl substituents of less than 2:1, or less than 1.9:1, or less than 1.8:1, or less than 1.7:1, or less than 1.6:1, or less than 1.5:1, or less than 1.4:1, or less than 1.3:1, or less than 1.2:1, or less than 1.1:1, or less than 1:1.

In one embodiment or in combination with any other embodiment, class or subclass of this third aspect, wherein the mixed cellulose ester has a ratio of hydroxyl substituents to butyryl substituents of at least 0.4:1, or at least 0.5:1, or at least 0.6:1, or at least 0.7:1, or at least 0.8:1, or at least 0.9:1, or at least 1:1, or at least 1.1:1, or at least 1.2:1, or at least 1.3:1, or at least 1.4:1, or at least 1.5:1, or at least 1.6:1, or at least 1.7:1, or at least 1.8:1, or at least 1.9:2, or at least 2:1. Additionally, or in the alternative, the mixed cellulose ester has a ratio of hydroxyl substituents to butyryl substituents of less than 2:1, or less than 1.9:1, or less than 1.8:1, or less than 1.7:1, or less than 1.6:1, or less than 1.5:1, or less than 1.4:1, or less than 1.3:1, or less than 1.2:1, or less than 1.1:1, or less than 1:1.

In one embodiment or in combination with any other embodiment, wherein the mixed cellulose ester is a cellulose acetate butyrate, or a cellulose acetate butyrate, or a mixture thereof. In one class of this embodiment, the mixed cellulose ester is a cellulose acetate butyrate. In one class of this embodiment, the mixed cellulose ester is a cellulose acetate propionate. In one class of this embodiment, the mixed cellulose ester is a mixture comprising a cellulose acetate butyrate or a cellulose acetate propionate.

In one embodiment or in combination with any other embodiment, class or subclass of this third aspect, wherein the MCE exhibits at least 40% biodegradability, or at least 45% biodegradability, or at least 50% biodegradability, or at least 55% biodegradability, or at least 60% biodegradability, or at least 65% biodegradability, or at least 70% biodegradability, or at least 75% biodegradability, or at least 80% biodegradability, or at least 85% biodegradability, or at least 90% biodegradability, or at least 90% biodegradability, at 56 days according to at least one of the OECD 301B, OECD 301C, or OECD 301F test methods.

In one embodiment or in combination with any other embodiment, class or subclass of this third aspect, wherein the MCE exhibits at least 40% biodegradability, or at least 45% biodegradability, or at least 50% biodegradability, or at least 55% biodegradability, or at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability at 60 days according to at least one of the OECD 301B, OECD 301C, or OECD 301F test methods.

In one embodiment or in combination with any other embodiment, class or subclass of this third aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

In one embodiment or in combination with any embodiment mentioned herein, the CE 100 can be the mixed cellulose ester of the first aspect, the second aspect, and/or the third aspect, including any class or subclass of these aspects.

### Solvent System

The solvent system, such as solvent 102, is generally capable of solubizing CE to produce the dispersed/solid phase of an emulsion/dispersion as described herein. In one embodiment or in combination with any embodiment mentioned herein, solvent 102 may consist of a single solvent component, or may be a solvent system including a plurality of solvent components. The plurality of solvent components may include at least two solvent components, at least three solvent components, or three total solvent components.

In one embodiment or in combination with any embodiment mentioned herein, solvent 102 includes at least one, at least two, or all three of a C1-C4 alkyl acetate, a C1-C4 alcohol, and water. The C1-C4 alkyl acetate may include one or more of methyl acetate, ethyl acetate, n-propyl acetate, isopropyl acetate, n-butyl acetate, isobutyl acetate, and sec-butyl acetate. The C1-C4 alcohol may include one or more of methanol, ethanol, propanols (e.g., isopropanol, n-propanol, and isopropyl alcohol), and butanols (e.g., n-butanol, isobutanol, sec-butanol, and tert-butanol).

In one embodiment or in combination with any embodiment mentioned herein, when solvent 102 includes more than one solvent component, the C1-C4 alkyl acetate may be present in one or more of the following amounts: (1) at least 10, or 25, 50, or 60, or 70 weight percent; (2) not more than 99, or 95, or 90, or 85, or 80 weight percent; and (3) in the range of 10-99, or 25-95, or 50-90, or 70-85 weight percent.

In one embodiment or in combination with any embodiment mentioned herein, when solvent 102 includes more than one solvent component, the C1-C4 alcohol is present in one or more of the following amounts: (1) at least 1, or 2, or 4, or 6, or 8, or 10 weight percent; (2) not more than 80, or 60, or 40, or 30, or 20, or 15 weight percent; and (3) in the range or 1-80, or 2-60, or 4-40, or 6-30, or 8-20, or 10-15 weight percent.

In one embodiment or in combination with any embodiment mentioned herein, when solvent 102 includes more than one solvent component, the water is present in one or more of the following amounts: (1) at least 1, or 2, or 4, or 6, or 8 weight percent; (2) not more than 50, or 25, or 20, or 15 weight percent; and (3) in the range of 1-50, or 2-25, 4-30, or 6-20, or 8-15 weight percent.

In one embodiment or in combination with any embodiment mentioned herein, solvent 102 includes at least one, at least two, or all three of ethyl acetate, n-propanol, and water. In such an embodiment, ethyl acetate is present in an amount in the range of 50-90, or 55-90, or 60-90, or 65-90, or 65-85, or 70-85, or 75-85 weight percent, n-propanol is present in an amount in the range of 4-40, or 5-35, or 6-30, or 7-25, or 8-20, or 10-20, or 10-15 weight percent, and water is present in an amount in the range of 4-30, or 5-25, or 6-20, or 7-15, or 8-12, or 9-12 weight percent.

Surprisingly, it has been found that water contributes and/or enables certain mixed cellulose esters to be solubilized. Particularly, it has been found that water contributes and/or enables mixed cellulose esters having high degrees of biodegradability, such as those associated with DS_{OH} values greater than a determined threshold, to be solubilized. These findings are disclosed in the EXPERIMENTS section below.

### Hydrocolloid

Hydrocolloids as described herein, such as hydrocolloid 106, are used as a colloidal protector and/or viscosity builder. In one embodiment or in combination with any embodiment mentioned herein, hydrocolloid 106 is a lyophilic colloid. For example, hydrocolloid 106 may include at least one of a gelatin, a natural gum, a protein, or a cellulose derivative. The cellulose derivative may include one or both of methyl cellulose, and carboxy methyl cellulose.

The hydrocolloid, such as carboxy methyl cellulose, may be selected based on a desired viscosity of the resulting aqueous mixture. In one embodiment or in combination with any embodiment mentioned herein, a “low” viscosity hydrocolloid has a viscosity in a range between 10-50 cps, a “medium” viscosity hydrocolloid has a viscosity in a range between 400-800 cps, and a “high” viscosity hydrocolloid has a viscosity in a range between 1500-3000 cps.

### Surfactant System

In one embodiment or in combination with any embodiment mentioned herein, surfactant 108 includes two or more individual emulsifiers. The individual emulsifiers may be differentiated from each other based on their Hydrophilic-Lipophilic Balance (HLB) numbers. For example, when surfactant 108 includes two individual emulsifiers, the emulsifiers may include a lower-HLB emulsifier, and a higher-HLB emulsifier.

In one embodiment or in combination with any embodiment mentioned herein, the HLB number of the higher-HLB emulsifier is at least 6, or 8, or 10, or 12, or 14, or 16, or 18, and the HLB number of the lower-HLB emulsifier not more than 12, or 10, or 8, or 6, or 4.

In one embodiment or in combination with any embodiment mentioned herein, the HLB number of the higher-HLB emulsifier is greater than the HLB number of the lower-HLB emulsifier by at least one of the following: (1) at least 2, 4, 8, 10, 12, or 14; (2) not more than 25, or 20, or 15; or (3) in the range of 2-25, or 8-20, or 12-15.

In one embodiment or in combination with any embodiment mentioned herein, the lower-HLB emulsifier is a glycerol ester of stearic acid. In one embodiment or in combination with any embodiment mentioned herein, the higher-HLB emulsifier is a secondary alcohol ethoxylate.

In one embodiment or in combination with any embodiment mentioned herein, surfactant 108 further includes a third emulsifier. The third emulsifier has an HLB number greater than the HLB number of the lower-HLB emulsifier. In one embodiment or in combination with any embodiment mentioned herein, the third emulsifier is a polyethylene glycol ester of stearic acid.

When the surfactant 108 is formed from all three emulsifiers, the lower-HLB emulsifier and the third emulsifier may be present in a ratio that is at least 0.25:1, or 0.5:1, or 0.75:1, or 1:1, or 1.25:1, or 1.5:1, or 1.75:1, or 2:1 and/or not more than 5:1, or 4:1, or 3:1, or 2:1, or 1.75:1, or 1.5:1, or 1.25:1.

The lower-HLB emulsifier and the third emulsifier may define a combined emulsifier. In addition, the higher-HLB emulsifier and the combined emulsifier may be present in surfactant 108 in a ratio that is at least 0.25:1, or 0.5:1, or 0.75:1, or 1:1, or 1.25:1, or 1.5:1, or 1.75:1, or 2:1 and/or not more than 5:1, or 4:1, or 3:1, or 2:1, or 1.75:1, or 1.5:1, or 1.25:1.

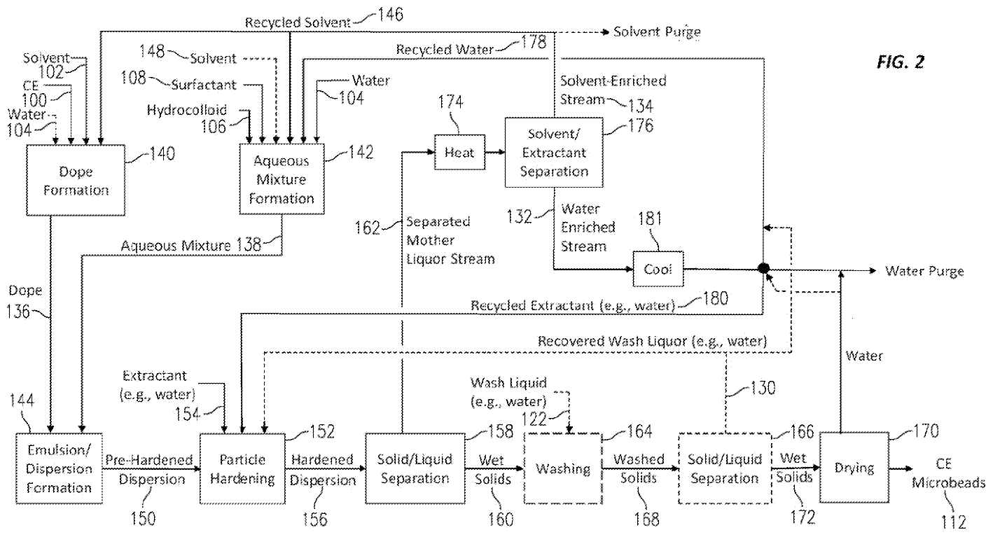

Referring now to FIG. 2, the illustrated process for making CE microbeads 112 includes separate formation of a CE dope 136 and an aqueous mixture 138. CE dope 136 and aqueous mixture 138 are then combined to form CE microbeads 112. For example, CE dope 136 may be formed at unit 140, and aqueous mixture 138 may be formed at unit 142. CE dope 136 and aqueous mixture 138 may then be combined at unit 144 to form an emulsion and/or dispersion, as will be described in more detail below. In one embodiment or in combination with any embodiment mentioned herein, CE dope 136 is formed from CE 100, solvent 102, water 104, and, in some embodiments, recycled solvent 146 derived from solvent-enriched stream 134.

In one embodiment or in combination with any embodiment mentioned herein, CE 100 is present in CE dope in one or more of: (1) at least 1, or 2, or 4, or 6, or 8, or 10 weight percent; (2) not more than 80, or 60, or 40, or 30, or 20, or 15 weight percent; and (3) in the range 1-80, or 2-60, or 4-40, or 6-30, or 8-20, or 10-15 weight percent.

In one embodiment or in combination with any embodiment mentioned herein, when solvent 102 includes a C1-C4 alkyl acetate, the C1-C4 alkyl acetate is present in CE dope 136 in one or more of the following amounts: (1) at least 10, 25, 50, 60, or 65 weight percent; (2) not more than 95, or 90, or 85, or 80 or 75 weight percent; and (3) in the range 10-95, or 25-90, or 50-85, or 65-75 weight percent.

In one embodiment or in combination with any embodiment mentioned herein, when solvent 102 includes a C1-C4 alcohol, the C1-C4 alcohol is present in CE dope 136 in one or more of the following amounts: (1) at least 1, or 2, or 4, or 6, or 8 weight percent; (2) not more than 50, or 25, or 20, or 15 weight percent; and (3) in the range of 1-50, or 2-25, or 4-30, or 6-20, or 8-15 weight percent.

In one embodiment or in combination with any other embodiment, the ratio of solvent to CE used in the dissolving to form CE dope 136 is at least 1:1, or 2:1, or 3:1, or 4:1, or 5:1 and/or not more than 100:1, or 50:1, or 25:1, or 10:1.

In one embodiment or in combination with any embodiment mentioned herein, when solvent 102 includes water, the water is present in CE dope 136 in one or more of the following amounts: (1) at least 0.5, or 1, or 2, or 4, or 6 weight percent; (2) not more than 40, or 25, or 15, or 10 weight percent; and (3) in the range of 0.5-40, or 1-25, or 2-15, or 4-20, or 6-10 weight percent.

In one particular embodiment, CE dope 136 is formed from CE 100, solvent 102 including ethyl acetate and n-propanol, and water 104. In such an embodiment, CE is present in CE dope 136 in an amount in the range of 6-30, or 7-25, or 8-20, or 9-20, or 10-15, or 12-15 weight percent, ethyl acetate is present in CE dope 136 in an amount in the range of 50-85, or 55-85, or 60-85, or 65-85, or 70-85, or 75-85 weight percent, n-propanol is present in CE dope 136 in an amount in the range of 4-30, or 6-25, or 8-20, or 10-20, or 12-15 weight percent, and water is present in CE dope 136 in an amount in the range of 4-20, or 5-18, or 6-16, or 7-14, or 8-12 weight percent.

In one embodiment or in combination with any embodiment mentioned herein, CE 100, solvent 102, and water 104 are combined at unit 140 until substantially homogeneous to produce CE dope 136. In one embodiment or in combination with any embodiment mentioned herein, these components are mixed at room temperature (e.g., at least 15, or 20, or 25, or 30° C. and/or not more than 45, or 40, or 35, or 30, or 25, or 20° C.) for a duration of at least 1, or 2, or 5, or 10, or 15, or 20, or 25, or 30, or 45, or 60, or 120, or 240 minutes and/or for as long as is needed to produce the substantially homogeneous mixture.

In one embodiment or in combination with any embodiment mentioned herein, aqueous mixture 138 is formed from water 104, hydrocolloid 106, surfactant 108, and, in some embodiments, recycled solvent 146 derived from solvent-enriched stream 134. Optionally, additional solvent 148 may be added to units 140 and/or 142 as needed to maintain concentration(s) of solvent components at suitable levels.

In one embodiment or in combination with any embodiment mentioned herein, the ratio of the recycled solvent portion to the fresh solvent portion used in said units 140 and/or 142 is at least 2.5:1, or 10:1, 25:1, or 50:1, or 75:1, or 90:1, or 95:1, or 99:1 by weight and/or not more 1000:1, or 500:1, or 200:1 or 100:1 by weight.

In one embodiment or in combination with any embodiment mentioned herein, the compositional make-up of the recycled solvent varies from the composition make-up of said fresh solvent by not more than 10, or 5, or 2, or 1 weight percent total.

In one embodiment or in combination with any embodiment mentioned herein, the fresh solvent and the recycled solvent portion have substantially the same composition.

In one embodiment or in combination with any embodiment mentioned herein, water is present in aqueous mixture 138 in one or more of the following amounts: (1) at least 40, or 60, or 70, or 80, or 85 weight percent; (2) not more than 99, or 97, or 95, or 94, or 92 weight percent; and (3) in the range of 40-99, or 70-95, or 85-92 weight percent.

In one embodiment or in combination with any embodiment mentioned herein, the hydrocolloid is present in aqueous mixture 138 in one or more of the following amounts: (1) at least 0.001, or 0.005, or 0.01, or 0.05, or 0.1 weight percent; (2) not more than 15, or 10, or 5, or 2, or 1 weight percent; and (3) in the range of 0.001-15, or 0.01-5, or 0.1-2 weight percent.

In one embodiment or in combination with any embodiment mentioned herein, the surfactant is present in aqueous mixture 138 in one or more of the following amounts: (1) at least 0.005, or 0.01, or 0.05, or 0.1, or 0.5 weight percent; (2) not more than 15, or 10, or 5, 2, or 1.5 weight percent; and (3) in the range of 0.005-15, or 0.05-5, or 0.5-1.5 weight percent.

As described above, the surfactant described herein may include a higher-HLB emulsifier and a lower-HLB emulsifier. In such embodiments, the higher-HLB emulsifier and the lower-HLB emulsifier are present in aqueous mixture 138 in a high-to-low HLB emulsifier ratio in the range of at least 0.25:1, or 0.5:1, 1:1, or 1.5:1, or 1.75:1 and/or not more than 10:1, or 5:1, or 3:1, or 2.5:1 and/or in the range of 0.25:1-10:1, or 0.5:1-5:1, or 1:1-3:1.

Referring again to FIG. 2, in some embodiments, additional solvent 148 and/or recycled solvent 146 is received at unit 142. In such embodiments, the solvent used to form CE dope 136 and aqueous mixture 138 is a common C1-C4 alkyl acetate. Using the at least one common component in the solvent systems received at units 140 and 142 facilitates simplification of separation, recovery, and re-use of the solvent as described herein.

In one embodiment or in combination with any embodiment mentioned herein, when the aqueous mixture 138 include a C1-C4 alkyl acetate, the C1-C4 alkyl acetate is present in aqueous mixture 138 in one or more of the following amounts: (1) at least 1, 2, 4, 6, or 8 weight percent; (2) not more than 50, 40, 30, 20, or 15 weight percent; and (3) in the range of 1-50, 2-40, or 6-20 weight percent.

In one embodiment or in combination with any embodiment mentioned herein, the C1-C4 alkyl acetate is present in aqueous mixture 138 in an amount, by weight percent, that is within 25, 20, 15, 10, 5, or 2 weight percent of the solubility, by weight percent, of the C1-C4 alkyl acetate in water at 20° C.

The C1-C4 alkyl acetate may be one or both of methyl acetate and ethyl acetate. In one embodiment or in combination with any embodiment mentioned herein, when the C1-C4 alkyl acetate is ethyl acetate, ethyl acetate is present in aqueous mixture in an amount in the range of 2-25, 4-20, 6-15, or 8-10 weight percent. In one embodiment or in combination with any embodiment mentioned herein, when the C1-C4 alkyl acetate is methyl acetate, methyl acetate is present in aqueous mixture in an amount in the range of 5-50, 10-40, 15-35, or 20-30 weight percent.

In one particular embodiment, aqueous mixture 138 is formed from water 104, hydrocolloid 106, surfactant 108, and a C1-C4 alkyl acetate. In such an embodiment, water is present in aqueous mixture 138 in an amount in the range of 70-95, 75-95, 80-95, 85-95, or 87.5-92.5 weight percent, the hydrocolloid is present in aqueous mixture 138 in an amount in the range of 0.01-5, 0.1-4, 0.5-3, 0.6-2, 0.7-1, or 0.8-0.9 weight percent, the surfactant is present in aqueous mixture 138 in an amount in the range of 0.05-5, 0.1-5, 0.1-4, 0.5-3, 0.6-2, 0.7-1, or 0.8-1 weight percent, and the C1-C4 alkyl acetate is present in aqueous mixture 138 in an amount in the range of 2-40, 3-35, 4-30, 5-25, 5-20, 5-15, 5-10, or 6-10 weight percent.

In one embodiment or in combination with any embodiment mentioned herein, water 104, hydrocolloid 106, surfactant 108, and optionally a C1-C4 alkyl acetate are combined at unit 142 to produce aqueous mixture 138. In one embodiment or in combination with any embodiment mentioned herein, these components are mixed at a temperature of at least 15, 20, 25, 30, 35, 40, 45, or 50° C. and/or not more than 100, 75, 50, 40, 35, 30, 25, or 20° C., for a duration of at least 1, 2, 5, 10, 15, 20, 25, 30, 45, 60, 120, or 240 minutes and/or for as long as is needed to produce a substantially homogeneous mixture, depending on the viscosity of the hydrocolloid used.

### Emulsion/Dispersion Formation

Once formed, CE dope 136 and aqueous mixture 138 may be combined at unit 144 to produce an emulsion and/or a dispersion, as described above.

In one embodiment or in combination with any embodiment mentioned herein, a ratio of CE dope 136 to aqueous mixture 138 combined at unit 144 to form the initial emulsion is at least 0.05:1 to 10:1, 0.1:1 to 5:1, 0.2:1 to 2:1, or 0.4:1 to 0.8:1.

In one embodiment or in combination with any embodiment mentioned herein, the initial emulsion comprises water in an amount of at least 10, 20, 30, 40, 50, or 60 weight percent and/or not more than 90, 80, 70, 60, 50, or 40 weight percent.

Once combined, the initial emulsion is converted into a pre-hardened dispersion including the solid phase and the liquid phase. This conversion may be carried out by at least one of shearing, spraying (ultrasonic or electro), and membrane emulsion.

In one embodiment or in combination with any embodiment mentioned herein, the combined CE dope and aqueous mixture is recirculated through a high shear mixer to disperse the solid phase within the liquid phase, and to facilitate hardening of the solid phase to produce the initial microparticles. This shearing may be performed as CE dope 136 and aqueous mixture 138 are fed to unit 144 and/or may be performed after predetermined quantities of CE dope 136 and aqueous mixture 138 are within unit 144.

In one embodiment or in combination with any embodiment mentioned herein, the combined CE dope and aqueous mixture is recirculated through the high shear mixer for at least 1, 2, 3, 4, or 5 residence times and/or for not more than 20, 15, 10, 9, or 8 residence times based on the total volume of the high shear mixer used. In addition, the high shear mixing is performed for a duration of at least 1, 2, 5, 10, 15, 20, 25, 30, 45, 60, 120, or 240 minutes and/or for as long as is needed to recirculate the volume of the mixture for the predetermined number of residence times.

To produce initial microparticles that will form the CE microbeads as described herein, agitation of the combined CE dope and aqueous mixture is performed at unit 144. The agitation performed at unit 144 may be quantified by at least one of the following: (1) impeller tip speed, (2) impeller Reynolds number, and (3) power to mass ratio. In one embodiment or in combination with any embodiment mentioned herein, the high shear mixing is performed at an impeller tip speed of at least 25, 50, 75, 100, 150, 200, 250, or 300 cm/s and/or not more than 1000, 500, 400, 300, 200, or 100 cm/s. In one embodiment or in combination with any embodiment mentioned herein, the high shear mixing is performed at an impeller Reynolds number of at least 500, 1000, 1500, 2000, 3000, 4000, 5000, or 6000 and/or not more than 15000, 10000, 8000, 6000, 5000, 4000, or 3000. In one embodiment or in combination with any embodiment mentioned herein, the high shear mixing is performed at a power to mass ratio of at least 0.01, 0.02, 0.03, 0.1, 0.5, 1.0, 1.5, 2.0, 2.5 or 3.0 and/or not more than 10.0, 7.5, 5.0, 4.0, 3.0, 2.5, 2.0, 1.5, 1.0 0.5, or 0.1.

As used herein, terms such as “solid,” “solid phase,” “particles,” and “microparticles” refer to semi-solid materials that do not lose their discrete nature (i.e., flowing together) when an aqueous/continuous phase surrounding the material is removed.

Hardening with Extractant

A pre-hardened dispersion 150 formed at unit 144, and including the initial microparticles (i.e., solid phase), may be channeled to a hardening unit 152 to convert the initial particles to hardened CE microbeads 112. At unit 152, the initial microparticles contained within pre-hardened dispersion 150 are contacted with an extractant 154 (i.e., a drowning liquid) to produce a hardened dispersion 156. That is, the contacting step facilitates de-solventizing and hardening the initial microparticles into CE microbeads. In one embodiment or in combination with any embodiment mentioned herein, the extractant is water. Alternatively, the extractant may be methanol, ethanol, and combinations thereof.

In one embodiment or in combination with any embodiment mentioned herein, the pre-hardened dispersion and the extractant are combined at unit 152 at an extractant-to-dispersion weight ratio of at least 0.5:1, 1:1, 1.5:1, 2:1, or 1.5:1 and/or not more than 10:1, 8:1, 6:1, 4:1, or 3:1.

In one embodiment or in combination with any embodiment mentioned herein, the weight ratio of extractant to pre-hardened particles used in the contacting step is at least 2:1, 5:1, 10:1, 20:1. 30:1, or 40:1 and/or not more than 200:1, 100:1, 80:1. 60:1, or 50:1 As a result, in one embodiment or in combination with any embodiment mentioned herein, hardened dispersion 156 has water in one or more of the following amounts: (1) at least 25, 50, 60, 70, 80, 85, or 90 weight percent; (2) not more than 99, 97.5, 95, 92.5, 90, 80, 70, 60, or 50 weight percent; and (3) in the range of 50-99, 70-95, or 80-92.5 weight percent.

In one embodiment or in combination with any embodiment mentioned herein, hardened dispersion 156 has water in a weight concentration that is at least 1.5, 2, 2.5, 3, 3.5, 4, 4.5, 5 times greater than and/or not more than 100, 70, 50, 25, 15, 10, 5, or 2.5 times greater than the weight concentration of water in the initial emulsion.

In one embodiment or in combination with any embodiment mentioned herein, hardening of the microparticles is carried out under agitation. The agitation performed at unit 152 may be quantified by at least one of the following: (1) impeller tip speed, (2) impeller Reynolds number, and (3) power to mass ratio. In one embodiment or in combination with any embodiment mentioned herein, the converting/hardening is performed at an impeller tip speed of at least 25, 50, 75, 100, 150, 200, 250, or 300 cm/s and/or not more than 1000, 500, 400, 300, 200, or 100 cm/s. In one embodiment or in combination with any embodiment mentioned herein, the converting/hardening is performed at an impeller Reynolds number of at least 500, 1000, 1500, 2000, 3000, 4000, 5000, or 6000 and/or not more than 15000, 10000, 8000, 6000, 5000, 4000, or 3000. In one embodiment or in combination with any embodiment mentioned herein, the converting/hardening is performed at a power to mass ratio of at least 0.01, 0.02, 0.03, 0.1, 0.5, 1.0, 1.5, 2.0, 2.5 or 3.0 and/or not more than 10.0, 7.5, 5.0, 4.0, 3.0, 2.5, 2.0, 1.5, 1.0 0.5, or 0.1.

In addition, the converting/hardening is performed for a time period of at least 0.1, 0.5, 1, 2, 4, 6, 8, 10, or 20 minutes and/or not more than 12, 8, 6, 4, or 2 hours, and at a temperature of at least 0, 5, or 10° C. and/or not more than 100, 75, 50, or 25° C.

In one embodiment or in combination with any embodiment mentioned herein, the converting/hardening is performed in a single vessel, or in multiple vessels. Multiple vessels may be needed based on the output of CE microbeads to be produced and the volumetric capacity of available vessels used for the hardening. In embodiments where multiple vessels are used, a first portion of pre-hardened dispersion 150 may be received at a first hardening unit, and a second portion of pre-hardened dispersion may be received at a second hardening unit. Flow communication may then be provided between the separate hardening units to enhance mass transfer such that desolventization of the initial microparticles is accelerated.

In one embodiment or in combination with any embodiment mentioned herein, pre-hardened dispersion 150 has a solids content of at least 0.5, 1, 2, 3, or 4 and/or not more than 40, 30, 20, 10, or 6 weight percent.

As a result of the hardening, in one embodiment or in combination with any embodiment mentioned herein, hardened dispersion 156 has a solids content of at least 0.05, 0.1, 0.5, or 1 weight percent and/or not more than 20, 10, 5, 2, or 1 weight percent In one embodiment or in combination with any embodiment mentioned herein, the solids content of the pre-hardened dispersion is at least 1.5, 2, 3, or 4 and/or not more than 20, 10, 8, or 6 times greater than the solids content of the hardened dispersion. In other words, at unit 152, solvent is extracted from the initial microparticles to produce hardened dispersion 156 including hardened CE microbeads 112 and a solvent-laden drowning liquid.

### Ce Microbead Isolation/Separation

CE microbeads 112 may be isolated and recovered from hardened dispersion 156 using any suitable technique at unit 158. In one embodiment or in combination with any embodiment mentioned herein, the isolating may be performed by at least one, at least two, or all three of the following: (1) flashing one or more liquid components away from the hardened CE microbeads; (2) filtering the hardened CE microbeads away from one or more liquid components; and (3) centrifuging, redispersing, and drying the hardened microbeads.

Solids processing and/or CE microbead isolation may be performed in a single unit, as illustrated in FIG. 1, or may be performed in multiple units, as illustrated in FIG. 2. Referring to FIG. 2, a wet solids stream 160 and a separated mother liquor stream 162 are discharged from unit 158. Wet solids stream 160 contains the hardened CE microbeads and residual liquid. In one embodiment or in combination with any embodiment mentioned herein, wet solids stream 160 has a solids content of at least 10, 20, 25, 30, 35, 40, or 45 weight percent and/or not more than 60, 65, 50, 45, or 40 weight percent. This enables wet solids stream 160 to be conveyed to downstream units with reduced clogging and processing concerns.

Wet solids stream 160 may then, optionally, be processed in a wash unit 164, and a second solid/liquid separation unit 166. At unit 164, the hardened CE microbeads are washed with water 122 to produce a washed solids stream 168. Washed solids stream 168 is then processed at unit 166 by at least one, at least two, or all three of the following: (1) flashing one or more liquid components away from the hardened CE microbeads; (2) filtering the hardened CE microbeads away from one or more liquid components; and (3) centrifuging, redispersing, and drying the hardened microbeads. This second solid/liquid separation step facilitates reducing the solvent content in the liquid around the hardened CE microbeads.

A wash liquor 130 may be recovered from the second solid/liquid separation step and then recycled for use as at least a portion of the aqueous mixture in unit 142 and/or for use as at least a portion of the drowning liquid in unit 152.

In one embodiment or in combination with any embodiment mentioned herein, at least 1, 5, or 10 weight percent and/or not more than 90, 50, 20, or 10 weight percent of aqueous mixture 138 is the recycled wash liquor.

In one embodiment or in combination with any embodiment mentioned herein, at least 1, 5, or 10 weight percent and/or not more than 90, 50, 20, or 10 weight percent of drowning liquid 154 used in unit 152 is the recycled wash liquor.

A wet solids stream 172 discharged from unit 166 is then received at a drying unit 170. In one embodiment or in combination with any embodiment mentioned herein, drying is performed at unit 170 under agitation and with the addition of heat. Such agitation facilitates reducing agglomeration of the recovered CE microbeads 112. Properties of the recovered CE microbeads 112 are described in more detail below.

In one embodiment or in combination with any embodiment mentioned herein, drying unit 170 is a rotary cone dryer.

### Liquids Processing and Recycle

Separated mother liquor stream 162 discharged from unit 164 may be processed to recover water and/or solvent. The recovered water and/or solvent may then be recycled to one or more of the units shown in FIG. 2 to enhance the economic efficiency of the microbeads formation processes described herein.

The liquid processing may be performed in a single unit, as illustrated in FIG. 1, or may be performed in multiple units, as illustrated in FIG. 2. Referring to FIG. 2, mother liquor stream 162 contains at least a portion of the water and at least a portion of the solvent introduced at units 140 and/or 142, for example. Mother liquor stream 162 may also contain residual amounts of the hydrocolloid, surfactant, and any components used in the making of the mixed cellulose esters.

Mother liquor stream 162 may be heated 174 and then separated at unit 176 into at least two separate streams, such as solvent-enriched stream 134 and water-enriched (solvent-depleted) stream 132. Mother liquor stream 162 may be separated using any suitable technique at unit 176. In one embodiment or in combination with any embodiment mentioned herein, the liquids separation may be performed by distillation and the like.

In one embodiment or in combination with any embodiment mentioned herein, water-enriched stream 132 includes one or more of the following: (1) water; (2) a surfactant; (3) a hydrocolloid; and (4) a C1-C4 alkyl acetate.

Accordingly, water-enriched stream 132 may be cooled 181 and then recycled to one or more units illustrated in FIG. 2. For example, the composition of water-enriched stream 132 enables it to be recycled to at least one of unit 142 as stream 178 for use in the forming of aqueous mixture 138, and/or to unit 152 as stream 180 for use in de-solventizing the initial microparticles, thereby reducing water usage required for such processes. Any excess not required by these processes may be purged from the system and subjected to wastewater treatment, for example.

In one embodiment or in combination with any embodiment mentioned herein, the ratio of recycled water in stream 180 used in the hardening step to recycled water in stream 178 used in forming aqueous mixture 138 is at least 1:1, 1.5:1, 2:1, 3:1, 4:1 and/or not more than 20:1, 10:1, 8:1, or 6:1.

In one embodiment or in combination with any embodiment mentioned herein, the ratio of recycled water in stream 180 used in the hardening step to purge water is at least 1:1, 1.5:1, 2:1, 3:1, 4:1 and/or not more than 20:1, 10:1, 8:1, or 6:1.

In one embodiment or in combination with any embodiment mentioned herein, at least 75, 90, 95, 98, 99, or 100 weight percent of the extractant used in unit 152 is recycled water recovered downstream from unit 152, such as water contained in water-enriched stream 132 that is recycled to unit 152.

In one embodiment or in combination with any embodiment mentioned herein, fresh water 154 is added to unit 152 for use in the hardening step.

In one embodiment or in combination with any embodiment mentioned herein, wherein the ratio of the total amount of added fresh water to purge water is at least 0.25:1 0.5:1, 0.75:1, or 0.9:1 and/or not more than 4:1, 2:1, 1.5:1, 1.25:1, or 1.1:1.

In one embodiment or in combination with any embodiment mentioned herein, solvent-enriched stream 134 includes one or more of: (1) a C1-C4 alkyl acetate; (2) a C1-C4 alcohol; and (3) water.

Accordingly, solvent-enriched stream 134 may be recycled to one or more units illustrated in FIG. 2. For example, the composition of solvent-enriched stream 134 enables it to be recycled to at least one of unit 140 for use as a portion of the solvent in the forming of CE dope 136, and/or to unit 142 for use in the forming of aqueous mixture 138. Any excess not required by these processes may be purged from the system.

In one embodiment or in combination with any embodiment mentioned herein, at least 75, 90, 95, 98, 99, or 100 weight percent of the solvent used in unit 140 used to form CE dope 136 is recycled solvent 146.

In one embodiment or in combination with any embodiment mentioned herein, fresh water 104 is added to unit 142 for use in forming aqueous mixture 138.

In one embodiment or in combination with any embodiment mentioned herein, the ratio of the combined amount of water in streams 178 and 180 to the total amount of added fresh water 104 in unit 142 is at least 2:1, 4:1, 6:1, or 8:1.

In one embodiment or in combination with any embodiment mentioned herein, aqueous mixture 138 includes an azeotrope of water and C1-C4 alkyl acetate, and/or of water and C1-C4 alcohol, derived from water-enriched stream 132.

Hydroxyl is generally a strong hydrogen bonder. Thus, the opportunity for hydrogen bonding increases as the DS_{OH} of a given substance increases. The cosolvent alcohol of the solvent system described herein is also a strong hydrogen bonder. Surprisingly however, the ability of the cosolvent alcohol to hydrogen bond was found to decrease as the number of carbons (i.e., C1-C4) increases. Accordingly, lower carbon number binary solvent systems that did not contain water were found to be capable of dissolving cellulose esters having a DS_{OH} greater than a biodegradability threshold (e.g., DS_{OH} of greater than 0.8). In contrast, higher carbon number binary solvent systems that did not contain water were found to not be capable of dissolving cellulose esters having a DS_{OH} greater than the same biodegradability threshold. Accordingly, it has been found that the presence of a strong hydrogen bonder (i.e., water) with essentially no steric hindrance in solvent systems containing higher carbon number alcohols facilitates dissolution of the mixed CEs described herein in the solvent systems.

In one embodiment or in combination with any embodiment mentioned herein, recycled solvent 146 contains water in an amount of at least 1, 2, 4, 6, or 8 weight percent and/or not more than 50, 40, 30, 20, or 10 weight percent.

In one embodiment or in combination with any embodiment mentioned herein, recycled solvent 146 contains at least one azeotrope, wherein the azeotrope contains water and another component.

In one embodiment or in combination with any embodiment mentioned herein, the azeotrope may be in recycled solvent 146 may be a water/alcohol azeotrope, a water/alkyl acetate azeotrope, or both a water/alcohol azeotrope and a water/alkyl acetate azeotrope.

In one embodiment or in combination with any embodiment mentioned herein, recycled solvent 146 contains a plurality of azeotropes. In one embodiment or in combination with any embodiment mentioned herein, the plurality of azeotropes include a plurality of binary azeotropes. In one embodiment or in combination with any embodiment mentioned herein, the components of the solvent system are selected such that recycled solvent 146 does not contain any ternary azeotropes, thereby simplifying recovery and recycle of said solvent.

In one embodiment or in combination with any embodiment mentioned herein, solvent-enriched stream 134 contains less than 10 weight percent, less than 1 weight percent, less than 0.1 weight percent, or 0.0 weight percent of a ternary azeotrope.

In one embodiment or in combination with any embodiment mentioned herein, recycled solvent 146 contains three total binary azeotropes, such as a water/alcohol azeotrope, a water/alkyl acetate azeotrope, and an alcohol/alkyl acetate azeotrope.

### Additional Embodiments

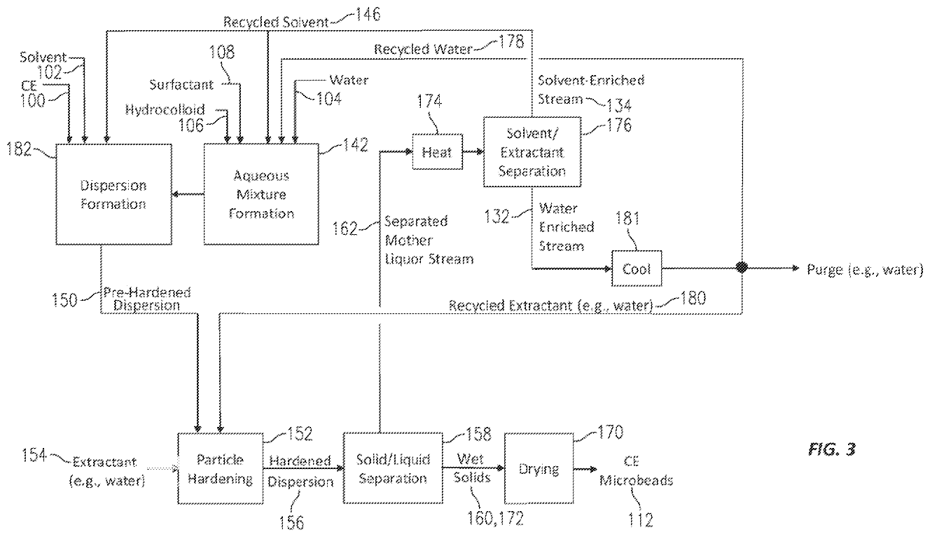

Referring now to FIG. 3, CE microbeads 112 are produced by serialized particle formation. In the example embodiment, particle formation begins at unit 142, in which water 104, hydrocolloid 106, surfactant 108, and, in some embodiments, recycled solvent 146 derived from solvent-enriched stream 134 is combined to form aqueous mixture 138.

Aqueous mixture 138 is discharged from unit 142 and received at a dispersion formation unit 182. At unit 182, aqueous mixture 138 is combined with CE 100, solvent 102, and, optionally, recycled solvent 146 derived from solvent-enriched stream 134. Thus, rather than forming CE dope 136 and aqueous mixture 138 in separate units, aqueous mixture 138, CE 100, and solvent 102 are combined in a common unit to form the initial emulsion.

In one embodiment or in combination with any embodiment mentioned herein, aqueous mixture 138, CE 100, and solvent 102 are agitated in unit 182 the combined CE dope and aqueous mixture is recirculated through a high shear mixer to disperse the solid phase within the liquid phase of the initial emulsion, and to facilitate hardening of the solid phase to produce the initial microparticles.

To produce the initial microparticles, agitation of the combined aqueous mixture 138, CE 100, and solvent 102 is performed at unit 182. The agitation performed at unit 182 may be quantified by at least one of the following: (1) impeller tip speed, (2) impeller Reynolds number, and (3) power to mass ratio. In one embodiment or in combination with any embodiment mentioned herein, the high shear mixing is performed at an impeller tip speed of at least 25, 50, 75, 100, 150, 200, 250, or 300 cm/s and/or not more than 1000, 500, 400, 300, 200, or 100 cm/s. In one embodiment or in combination with any embodiment mentioned herein, the high shear mixing is performed at an impeller Reynolds number of at least 500, 1000, 1500, 2000, 3000, 4000, 5000, or 6000 and/or not more than 15000, 10000, 8000, 6000, 5000, 4000, or 3000. In one embodiment or in combination with any embodiment mentioned herein, the high shear mixing is performed at a power to mass ratio of at least 0.01, 0.02, 0.03, 0.1, 0.5, 1.0, 1.5, 2.0, 2.5 or 3.0 and/or not more than 10.0, 7.5, 5.0, 4.0, 3.0, 2.5, 2.0, 1.5, 1.0 0.5, or 0.1.

Pre-hardened dispersion 150 is then discharged from unit 182 and hardened CE microbeads 112 recovered therefrom as described above.

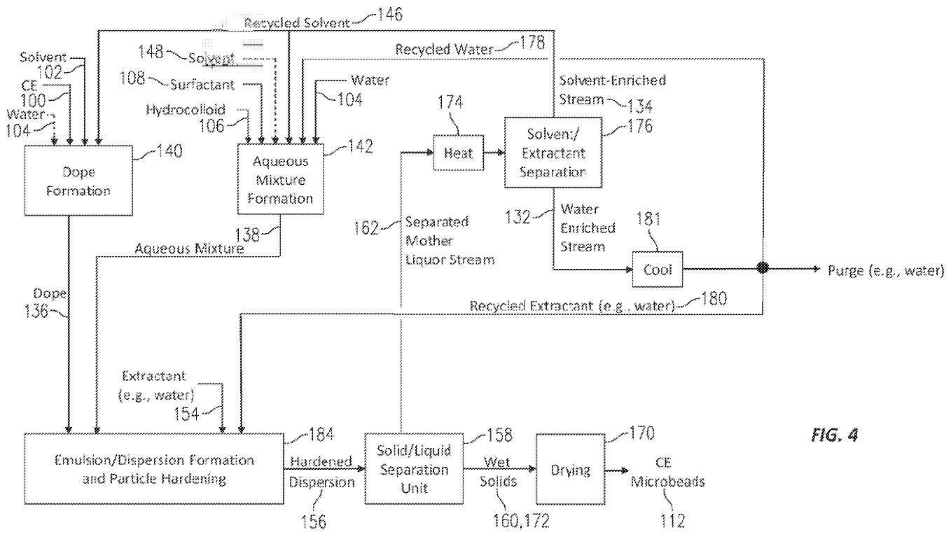

Referring to FIG. 4, CE microbeads 112 are produced by separated dope and aqueous mixture formation, and combined emulsion/dispersion formation and particle hardening. In the example embodiment, particle formation begins as illustrated in FIG. 2, wherein CE dope 136 and aqueous mixture 138 are formed in separate units.

In FIG. 4 however, CE dope 136 and aqueous mixture 138 are fed to a common emulsion/dispersion formation and particle hardening unit 184. At unit 184, CE dope 136 and aqueous mixture 138 are combined and agitated to form the initial emulsion as described herein. Once the initial emulsion is formed, extractant 154 is fed directly to unit 184 to perform de-solventization of the initial microparticles.

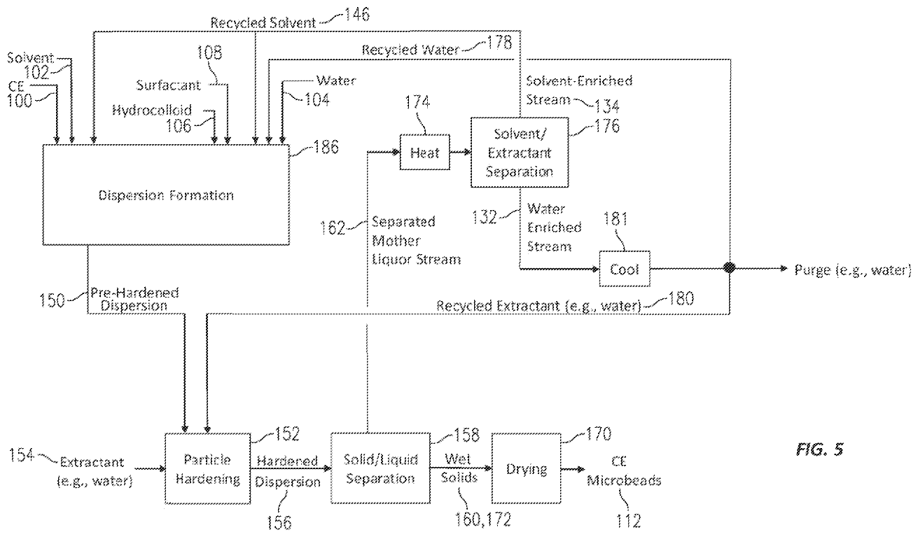

Referring to FIG. 5, CE microbeads 112 are produced by combined emulsion/dispersion formation. In the example embodiment, particle formation begins by combining all of CE 100, solvent 102, water 104, hydrocolloid 106, surfactant 108, and, optionally, recycled solvent 146 and recycled water 178 in a common unit 186. This mixture is agitated as described herein to produce pre-hardened dispersion 150 containing initial microparticles. Pre-hardened dispersion is received at unit 152 to de-solventize the initial microparticles as described herein.

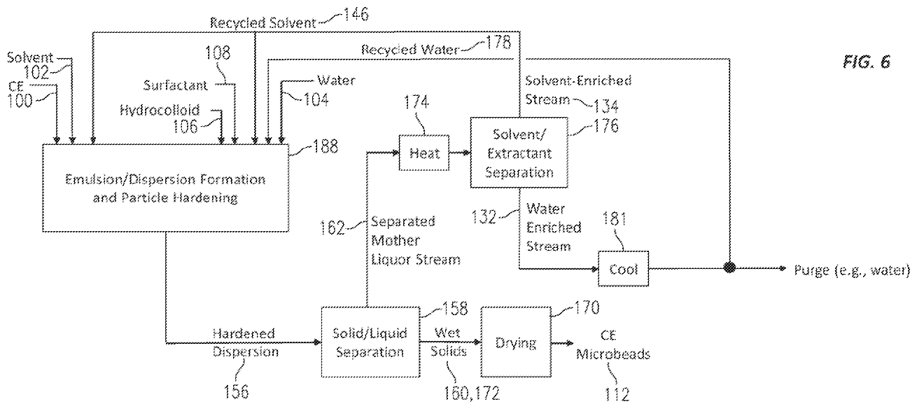

Referring to FIG. 6, CE microbeads 112 are produced by combined emulsion/dispersion formation and particle hardening. In the example embodiment, particle formation begins by combining all of CE 100, solvent 102, water 104, hydrocolloid 106, surfactant 108, and, optionally, recycled solvent 146 and recycled water 178 in a common unit 188. At unit 188, these components are combined and agitated to form the initial emulsion as described herein. Once the initial emulsion is formed, extractant 154 is fed directly to unit 188 to perform de-solventization of the initial microparticles.

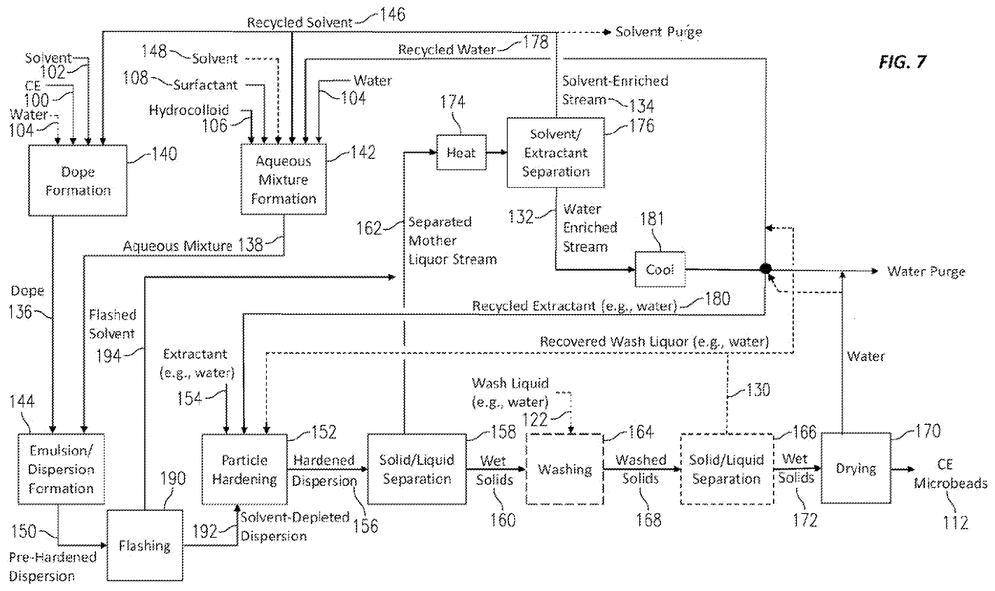

Referring to FIG. 7, CE microbeads 112 are produced by solvent flashing prior to particle hardening. In the example embodiment, pre-hardened dispersion 150 discharged from unit 144 is received at a flash unit 190, rather than at particle hardening unit 152. At unit 144, at least some of the solvent of pre-hardened dispersion 150 is removed from the liquid phase thereof, to thereby form a solvent-depleted dispersion 192 having a reduced solvent content. Solvent-depleted dispersion 192 is received at unit 152 to harden the pre-hardened microparticles contained therein, as described above. A flashed solvent stream 194 discharged from unit 190 may be channeled to unit 176 to perform liquids processing and recycle thereof.

In one embodiment or in combination with any embodiment mentioned herein, the removing at unit 144 is performed pervaporation, crossflow membrane filtration (ultrafiltration or nanofiltration), flash pot, spray pot, or wiped film evaporation.

In one embodiment or in combination with any embodiment mentioned herein, the removing at unit 144 reduces the solvent in said dispersion by at least 30 percent, at least 50 percent, at least 75 percent, at least 90 percent, between 30 and 90 percent, or between 50 and 75 percent by weight.

In one embodiment or in combination with any embodiment mentioned herein, solvent-depleted dispersion 192 has a solids content of at least 3, 4, 5, or 6 and/or not more than 40, 30, 20, or 10 weight percent.

In one embodiment or in combination with any embodiment mentioned herein, a volume ratio of drowning liquid to said dispersion used in unit 152 is less than 2.5:1, 2:1, 1.75:1, 1.5:1, 1.25:1, or 1:1.

### Hardened or Jet Milled Ce Microbeads

The hardened or jet milled CE microbeads produced by the processes disclosed herein exhibit desirable tactile and/or optical qualities, for example, making them desirable for use in personal care products, cosmetics, and the like. As used herein, the terms “microparticles,” “beads,” and “microbeads” may be used interchangeably with the term “hardened CE microbeads” or “jet milled CE microbeads.”

In one embodiment or in combination with any embodiment mentioned herein, CE microbeads are produced at a rate of at least 50, 100, 250, 500, 1000, 2500, 5000, or 10000 kg/day and/or not more than 100000, 75000, or 50000 kg/day. To achieve these production rates, CE, solvent, and/or water is provided within the system at one or more of the following corresponding rates.

In one embodiment or in combination with any embodiment mentioned herein, CE is provided at unit 140 at a rate of at least 50, 100, 250, 500, 1000, 2500, 5000, or 10000 kg/day and/or not more than 100000, 75000, or 50000 kg/day.

In one embodiment or in combination with any embodiment mentioned herein, solvent is provided at unit 140 at a rate of at least at least 50, 100, 250, 500, 1000, 2500, 5000, or 10000 kg/day and/or not more than 100000, 75000, or 50000 kg/day.

In one embodiment or in combination with any embodiment mentioned herein, water is provided unit 152 at a rate of at least at least 500, 1000, 2500, 5000, 10000, 25000, 50000, or 100000 kg/day and/or not more than 1000000, 750000, or 500000 kg/day.

In one embodiment or in combination with any embodiment mentioned herein, the hardened CE microbeads have a hardness at 20° C. that is greater than the hardness at 20° C. of the initial microparticles contained within pre-hardened dispersion 150 and/or formed in any of the processes described herein.

In one embodiment or in combination with any embodiment mentioned herein, the hardened CE microbeads have a hardness of at least 1.1, 1.25, 1.5, 1.75, or 2 times greater than the hardness of the initial microparticles.

In one embodiment or in combination with any embodiment mentioned herein, the hardened CE microbeads have a solvent content of less than 100, 50, 25, or 10 ppm.

In one embodiment or in combination with any embodiment mentioned herein, the hardened or jet milled CE microbeads have a solvent content of less than the solvent content of the initial microparticles.

In one embodiment or in combination with any embodiment mentioned herein, the hardened or jet milled CE microbeads have a solvent content of less than 0.99, 0.95, 0.9, 0.8, 0.7, 0.6, or 0.5 of the solvent content of the initial microparticles.

In one embodiment or in combination with any embodiment mentioned herein, the hardened or jet milled CE microbeads have a solvent content that is at least 1, 2, 4, 8, 12, 16 or 20 weight percent less than the solvent content of the initial microparticles.

In one embodiment or in combination with any embodiment mentioned herein, the hardened or jet milled CE microbeads have a D50 volume-based particle size that is within 50, 25, 15, 10, 5, or 2 percent of the D50 particle size of the initial microparticles.

In one embodiment or in combination with any embodiment mentioned herein, the hardened or jet milled CE microbeads have a D50 volume-based particle size of less than 0.99, 0.95, 0.9, 0.8, 0.7, 0.6, or 0.5 of the D50 volume-based particle size of the initial microparticles.

In one embodiment or in combination with any embodiment mentioned herein, the hardened or jet milled CE microbeads have a D50 volume-based particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 2 to 100, 2 to 80, 2 to 70, 2 to 60, 2 to 50, 2 to 40, 2 to 35, 2 to 30, 2 to 25, 2 to 20, 2 to 15, 2 to 10, 3 to 100, 3 to 80, 3 to 70, 3 to 60, 3 to 50, 3 to 40, 3 to 35, 3 to 30, 3 to 25, 3 to 20, 3 to 15, 3 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the hardened or jet milled CE microbeads can have a D50 volume-based particle size of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 microns.

As used herein, the term “D50 volume-based” means that 50% of the beads have a maximum dimension that is less than or equal to the noted value (e.g., 10 microns), based on a volume basis. The D50 value may also be treated as the median particle size. To ensure that a representative D50 value is obtained, the sample size of the beads should be at least 0.5 grams. The microbead sample size is then dispersed and mixed in 1.5 ounces of isopropanol or aqueous surfactant solution (1 drop 5% v/v Igepal® CO-630 surfactant). Testing for D50 is performed via laser diffraction and computer algorithms using the Mie theory to generate a particle size distribution. One suitable particle size analyzer for determining D50 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the hardened or jet milled CE microbeads have a D10 volume-based particle size of 0.5 to 20, 0.5 to 15, 0.5 to 12, 0.5 to 10, 0.5 to 5, 0.5 to 4, 0.5 to 3, 0.5 to 2, 0.5 to 1, 1 to 20, 1 to 15, 1 to 12, 1 to 5, 1 to 3, 2 to 20, 2 to 10, 2 to 5, 3 to 20, 3 to 15, 3 to 10, 4 to 20, 4 to 15, 4 to 10, 5 to 20, 5 to 15, 5 to 10, 10 to 20, or 10 to 15 microns. For example, the hardened or jet milled CE microbeads can have a D10 volume-based particle size of 0.5, 1, 2, 3, 4, 5, 6, 7, 8, 9, 10, 11, 12, 13, 14, 15, 16, 17, 18, 19, or 20 microns.

As used herein, the term “D10 volume-based” means that 10% of the beads have a maximum dimension that is less than or equal to the noted value (e.g., 10 microns), based on a volume basis. To ensure that a representative D10 value is obtained, the sample size of the beads should be at least 0.5 grams. The microbead sample size is then dispersed and mixed in 1.5 ounces of isopropanol or aqueous surfactant solution (1 drop 5% v/v Igepal® CO-630 surfactant). Testing for D10 is performed via laser diffraction and computer algorithms using the Mie theory to generate a particle size distribution. One suitable particle size analyzer for determining D10 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the hardened or jet milled CE microbeads have a D90 volume-based particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the hardened or jet milled CE microbeads can have a D90 volume-based particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D90 volume-based” means that 90% of the beads have a maximum dimension that is less than or equal to the noted value (e.g., 10 microns), based on a volume basis. To ensure that a representative D90 value is obtained, the sample size of the beads should be at least 0.5 grams. The microbead sample size is then dispersed and mixed in 1.5 ounces of isopropanol or aqueous surfactant solution (1 drop 5% v/v Igepal® CO-630 surfactant). Testing for D90 is performed via laser diffraction and computer algorithms using the Mie theory to generate a particle size distribution. One suitable particle size analyzer for determining D90 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the hardened or jet milled CE microbeads have a D100 volume-based particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the hardened or jet milled CE microbeads can have a D100 volume-based particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D100 volume-based” means that 100% of the beads have a maximum dimension that is less than or equal to the noted value (e.g., 10 microns), based on a volume basis. To ensure that a representative D100 value is obtained, the sample size of the beads should be at least 0.5 grams. The microbead sample size is then dispersed and mixed in 1.5 ounces of isopropanol or aqueous surfactant solution (1 drop 5% v/v Igepal® CO-630 surfactant). Testing for D100 is performed via laser diffraction and computer algorithms using the Mie theory to generate a particle size distribution. One suitable particle size analyzer for determining D100 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (˜4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the hardened or jet milled CE microbeads have an average sphericity of at least 10, or at least 20, or at least 30, or at least 40, or at least 50, or at least 60, or at least 65, or at least 70, or at least 75, or at least 80, or at least 85, or at least 90, or at least 95, or at least 96, or at least 97, or at least 98, or from 20 to 100, or from 20 to 90, or from 20 to 80, or from 20 to 70, or from 20 to 60, or from 30 to 100, or from 30 to 90, or from 30 to 80, or from 30 to 70, or from 30 to 60, or from 40 to 100, or from 40 to 90, or from 40 to 80, or form 40 to 70, or from 40 to 60, or from 50 to 100, or from 50 to 90, or from 50 to 80, or from 50 to 70, or from 50 to 60, or from 60 to 100, or from 60 to 90, or from 60 to 80, or from 60 to 70, or from 70 to 100, or from 70 to 90, or from 70 to 80, or from 80 to 100, or from 80 to 90, or from 90 to 100 percent. Additionally, or in the alternative, the hardened or jet milled CE microbeads can have an average sphericity of not more than 99, 95, 90, 80, 70, 60, 50, 40, or 30 percent.

Average sphericity is determined by: (1) obtaining a secondary emission/ETD detector scanning electron microscopy (SEM) image of a representative sample of at least 40 microbeads, (2) on the SEM image, selecting a square sample window centered at the center of the SEM that contains exactly 30 microbeads whose entire outer perimeters are clearly visible (i.e., not occluded), (3) measuring the maximum and minimum diameters (each extending through the particle's centroid and not necessarily perpendicular to one another) of the 30 clearly visible microbeads in the sample window, (4) for each of the 30 particles, dividing the minimum diameter by the maximum diameter and multiplying the result by 100% to obtain 30 individual particle sphericities, and (5) averaging the 30 individual particle sphericities to obtain the average sphericity.

In one embodiment or in combination with any embodiment mentioned herein, the hardened or jet milled CE microbeads exhibit a monomodal particle size distribution with a span of of at least 0.5, at least 0.55, at least 0.60, at least 0.65, at least 0.70, at least 0.75, at least 0.80, at least 0.85, at least 0.9, at least 0.95, at least 1.0, at least 1.05, at least 1.1, at least 1.15, at least 1.2, or at least 1.25. In one embodiment or in combination with any embodiment mentioned herein, the hardened or jet milled CE microbeads exhibit a monomodal particle size distribution with a span of at least 0.5, 0.55, 0.6, 0.65, 0.7, 0.75, 0.8, 0.85, 0.9, 0.95, 1.0, 1.05, 1.1, 1.15, 1.2, 1.25, 1.3, or 1.4 and/or less than 3.0, 2.9, 2.8, 2.7, 2.6, 2.5, 2.4, 2.3, 2.2, 2.1, 2.0, 1.9, 1.8, 1.7, 1.6, or 1.5. In certain embodiments, the hardened or jet milled CE microbeads exhibit a monomodal particle size distribution with a span of 1.0 to 3.0, 1.0 to 2.5, 1.0 to 2.0, 1.0 to 1.8, 1.0 to 1.6, 1.2 to 3.0, 1.2 to 2.5, 1.2 to 2.0, 1.2 to 1.8, 1.2 to 1.6, 1.3 to 3.0, 1.3 to 2.5, 1.3 to 2.0, 1.3 to 1.8, or 1.3 to 1.6. As used herein, “monomodal particle size distribution” refers to a particle size distribution for a material that only has a single notable peak of size distribution. This is in contrast to multi-modal particle size distributions, which will have two or more peaks of particle size distributions. The “span” of the monomodal peak may be measured using the D10, D50, and D90 values of the particles using the following formula:

(Dₓ(90)−Dₓ(10))/Dₓ(50),

wherein “x” is the designated particle size.

In one embodiment or in combination with any embodiment mentioned herein, the hardened or jet milled CE microbeads have an average smoothness of at least 10, 20, 30, 40, 50, 60, 70, 80, 90, 95, 95, 97, 98, or 99 percent. Additionally, or in the alternative, the hardened CE or jet milled microbeads can have an average smoothness of not more than 99, 95, 90, 80, 70, 60, 50, 40, or 30 percent.

Average smoothness is determined by: (1) obtaining a secondary emission/EDT detector scanning electron microscopy (SEM) image of a representative sample of at least 20 microbeads, (2) on the SEM, selecting a square sample window centered at the center of the SEM that contains exactly 10 microbeads whose entire outer perimeters are clearly visible (i.e., not occluded), (3) binarizing the sample window by manual binarization with upper and lower thresholds chosen to match the exact shape of the darker regions of the particles, (4) for each of the 10 microbeads, selecting a square window at or near the center of the particle having length and width that are approximately ⅓ of the particle diameter (before binarizing the particle), (5) dividing the dark region area in the square window by the total area of the square window and multiplying the result by 100% to obtain 10 individual particle smoothnesses, and (5) averaging the 10 individual particle smoothnesses to obtain the average smoothness.

In one embodiment or in combination with any embodiment mentioned herein, the hardened or jet milled CE microbeads have an average BET surface area of at least 0.1, 0.5, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4. 6.5, 6.7, 6.8, 6.9, 7.0, 7.1, 7.2, 7.3, 7.4, 7.5, 7.6, 7.7, 7.8, 7.9, or 8.0 m²/g and/or not more than 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2.5, 2, 1.9, 1.8, 1.7, 1.6, 1.5, 1.4, or 1.3 m²/g as measured according to ISO 9277 using a Micromeritics ASAP 2020 instrument and krypton gas.

In one embodiment or in combination with any embodiment mentioned herein, the hardened or jet milled CE microbeads have a BET average pore size of at least 25, 30, 35, 40, 45, 50, 55, 56, 57, 58, or 59 angstroms and/or less than 75, 70, 65, or 60 angstroms as measured according to ISO 9277 and ISO 15901-02 using a Micromeritics ASAP 2020 instrument and nitrogen gas.

In one embodiment or in combination with any embodiment mentioned herein, the hardened or jet milled CE microbeads have a BJH average pore size of at least 50, 60, 70, 80, 90, 100, 110, 120, 125, or 130 angstroms and/or less than 200, 190, 180, 170, 160, 150, 140, or 130 angstroms as measured according to ISO 15901-02 using a Micromeritics ASAP 2020 instrument and nitrogen gas.

In one embodiment or in combination with any embodiment mentioned herein, the hardened or jet milled CE microbeads have a BJH surface area of pores from 17 to 3,000 angstroms of at least 0.5, 1.0, 1.1, 1.2, 1.3, 1.4. or 1.5 and/or less than 2.5, 2.0, 1.9, 1.8, 1.7, 1.6, 1.5, 1.4, or 1.3 m²/g as measured according to ISO 15901-02 using a Micromeritics ASAP 2020 instrument and nitrogen gas.

In one embodiment or in combination with any embodiment mentioned herein, the hardened or jet milled CE microbeads have a BJH volume of pores from 17 to 3,000 angstroms of at least 0.001, 0.002, 0.003, or 0.004 and/or less than 0.1, 0.05, or 0.01 mL/g as measured according to ISO 15901-02 using a Micromeritics ASAP 2020 instrument and nitrogen gas.

In one embodiment or in combination with any embodiment mentioned herein, the hardened or jet milled CE microbeads have a true specific gravity of at least 0.3, 0.4, 0.5, 0.6, 0.7, 0.8, or 0.9 and/or not more than 1.5, 1.4, 1.3, 1.2, 1.1, 1.0, 0.9, 0.8, 0.7, or 0.6 as measured by JIS Z8807-1976.

In one embodiment or in combination with any embodiment mentioned herein, the hardened or jet milled CE microbeads have a bulk specific gravity of at least 0.2, 0.3, 0.4, 0.5, 0.6, 0.7, or 0.8 and/or not more than 1.4, 1.3, 1.2, 1.1, 1.0, 0.9, 0.8, 0.7, 0.6, or 0.5 as measured by JIS 1201-1.

In one embodiment or in combination with any embodiment mentioned herein, the hardened or jet milled CE microbeads have a polydispersity index of less than 0.8, 0.7, 0.6, 0.5, 0.4, or 0.3.

In one embodiment or in combination with any embodiment mentioned herein, the hardened or jet milled CE microbeads have a surfactant content of less than 200, 150, 100, 50, 20, 10, 7.5, 5, 2.5, or 1 ppmw.

In one embodiment or in combination with any embodiment mentioned herein, the hardened or jet milled CE microbeads have a plasticizer content of less than 200, 150, 100, 50, 20, 10, 7.5, 5, 2.5, or 1 ppmw.

In one embodiment or in combination with any embodiment mentioned herein, the hardened or jet milled CE microbeads have a butyric acid content of less than 500, 400, 300, 200, 100, 50, 20, 10, 7.5, 5, 2.5, or 1 ppmw.

In one embodiment or in combination with any embodiment mentioned herein, the hardened or jet milled CE microbeads have an acetic acid content of less than 500, 400, 300, 200, 100, 50, 20, 10, 7.5, 5, 2.5, or 1 ppmw.

In one embodiment or in combination with any embodiment mentioned herein, the hardened or jet milled CE microbeads have a propionic acid content of less than 500, 400, 300, 200, 100, 50, 20, 10, 7.5, 5, 2.5, or 1 ppmw.

The butyric acid, acetic acid, and propionic acid contents of the hardened or jet milled CE microbeads may be measured via gas chromatography (“GC”). Under one GC methodology, the butyric acid, acetic acid, and propionic acid contents may be measured by adding about 100 mg of the hardened or jet milled CE microbeads to a tared 4-dram vial, followed by the addition of an internal standard solution comprising nonane in a 90:10 mixture of dichloromethane/methanol. A magnetic stir bar is placed in the vial, and the sample is stirred for two hours. After stirring, 8.0 mL of n-heptane is added dropwise to precipitate the polymer, and then the sample is vortexed. Approximately 100 mg of the supernatant is transferred to a GC vial, along with 100 μL of pyridine and 450 μL of BSTFA. The samples are heated at 80° C. for 30 minutes and then cooled to room temperature before injection. Samples are chromatographed simultaneously on 100% dimethylpolysiloxane and 14% cyanopropyl-phenyl-methylpolysiloxane columns using temperature programming and flame ionization detection. Alternatively, a second GC methodology involves preparing samples by adding approximately 30 mg of hardened or jet milled CE microbeads to a tared GC vial, followed by 200 μL of an internal standard solution comprising decane in pyridine, and 1.0 mL of BSTFA. The vials re heated at 80° C. for 30 minutes and then cooled to room temperature before injection. Samples are then chromatographed simultaneously on 100% dimethylpolysiloxane and 6% cyanopropyl-phenyl-methylpolysiloxane columns using temperature programming and flame ionization detection.

In one embodiment or in combination with any embodiment mentioned herein, the hardened or jet milled CE microbeads have a sulfuric acid content of less than 500, 400, 300, 200, 100, 50, 20, 10, 7.5, 5, 2.5, or 1 ppmw. The sulfuric acid content of the hardened or jet milled CE microbeads may be measured by the following methodology. First, the tested sample is added to a titration cell and dissolved in a solvent for a total volume of 70 mL. Solvent blanks are also prepared for comparison purposes. The samples and blanks are then titrated with 0.05 N potassium hydroxide in methanol using an automatic titrator equipped with a combination glass potentiometric electrode.

Acid number is calculated based on the sample weight and the KOH consumed in the sample minus the KOH consumed in the blank.

In one embodiment or in combination with any embodiment mentioned herein, the hardened or jet milled CE microbeads have a CE content of at least 50, 55, 60, 65, 70, 75, 80, 85, 90, 91, 92, 93, 94, 95, 96, 97, 98, or 99 weight percent of the mixed cellulose ester of the first aspect, the second aspect, and/or the third aspect, including any class or subclass of these aspects. Additionally, or in the alternative, the hardened or jet milled CE microbeads may have a CE content of less than 99.9, 99.5, 99, 98, 97, 96, 95, 94, 93, 92, 91, 90, 89, 88, 87, 86, or 85 weight percent of the mixed cellulose ester of the first aspect, the second aspect, and/or the third aspect, including any class or subclass of these aspects. In certain embodiments, the hardened or jet milled CE microbeads may consist essentially of the mixed cellulose ester of the first aspect, the second aspect, and/or the third aspect, including any class or subclass of these aspects.

In one embodiment or in combination with any embodiment mentioned herein, the hardened or jet milled CE microbeads may contain an additional biodegradable cellulose ester that is different from the mixed cellulose ester of the first aspect, the second aspect, and/or the third aspect. In such embodiments, this additional cellulose ester can be cellulose acetate, which exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability at 60 days according to at least one of the OECD 301B, OECD 301C, or OECD 301F test methods.

In one embodiment or in combination with any embodiment mentioned herein, the hardened or jet milled CE microbeads may contain at least 1, 2, 3, 4, 5, 6, 7, 8, 9, or 10 weight percent the additional biodegradable cellulose ester that is different from the mixed cellulose ester of the first aspect, the second aspect, and/or the third aspect. Additionally, or in the alternative, the hardened or jet milled CE microbeads may contain less than 50, 45, 40, 35, 30, 25, 20, 15, 10, or 5 weight percent of the additional biodegradable cellulose ester that is different from the mixed cellulose ester of the first aspect, the second aspect, and/or the third aspect.

In one embodiment or in combination with any embodiment mentioned herein, the hardened or jet milled CE microbeads have moisture content in one or more of the following amounts: (1) greater than 0 weight percent; (2) not more than 10, 9, 8, 7, 6, 5, 4, 3, 2, 1, or 0.05 weight percent; and (3) in the range of 0-10, 0-5, 0-4, 0-3, or 1.3 weight percent.

In one embodiment or in combination with any other embodiment mentioned herein, the hardened or jet milled CE microbeads exhibit at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability at 56 days according to at least one of the OECD 301B, OECD 301C, or OECD 301F test methods.

In one embodiment or in combination with any other embodiment mentioned herein, the hardened or jet milled CE microbeads exhibit at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, or at least 90% biodegradability, or at least 95% biodegradability at 60 days according to at least one of the OECD 301B, OECD 301C, or OECD 301F test methods.

In one embodiment or in combination with any other embodiment mentioned herein, the hardened or jet milled CE microbeads exhibit an oil absorption of at least 30, 35, 40, 45, 50, 55, 60, 65, 70, 75, 80, 85, 90, 95, or 100 mL per 100 g as measured using test method ASTM D281, wherein mineral oil is used instead of castor oil.

In one embodiment or in combination with any other embodiment mentioned herein, the hardened or jet milled CE microbeads exhibit a zeta potential of at least −95, at least −90, at least −85, at least −80, at least −75, at least −70, at least −65, at least −60, at least −55, at least −50, or at least −45 mV. Additionally, or in the alternative, the hardened or jet milled CE microbeads may exhibit a zeta potential of less than −5, less than −10, less than −15, less than −20, less than −25, less than −30, less than −35, less than −40, less than −45, less than −50, less than −55, less than −60, or less than −65 mV.

Zeta potential was measured by dispersing the microbeads in water by vortex mixing for 30 seconds. The microbead concentration was controlled at 0.5 mg/ml. Zeta potential tests were done on a Zetasizer Nano series, model ZEN 3600 instrument from Malvern Panalytical with a sample cell DTS1070. A Smoluchowski model is then used for Zeta potential calculation.

In one embodiment or in combination with any other embodiment mentioned herein, the hardened or jet milled CE microbeads exhibit a haze transmission at least 10, at least 15, at least 20, at least 25, at least 30, at least 35, at least 40, or at least 45 percent. Additionally, or in the alternative, the hardened or jet milled CE microbeads may exhibit a haze transmission less than 90, or less than 85, or less than 80, or less than 75, or less than 70, or less than 65, or less than 60, or less than 55, or less than 50, or less than 45, or less than 40 percent, or less than 35 percent, or less than 32 percent, or less than 30 percent. The haze transmission may be measured using a BYK Glossmeter and a BYK Haze Gard I by forming an aqueous emulsion comprising 5 weight percent or 7 weight percent of the biodegradable beads.

The detailed procedure for measuring the haze follows. The W/O emulsion for measuring haze % was prepared using the following procedure.

Two phases were used for preparing the emulsion. Phase A is made from water, magnesium sulfate heptahydrate (Merck), and Euxyl PE9010 (Ashland) at the following weight concentration, 59:2:1, respectively. Phase B is prepared with caprylic/capric triglycerides (Making Cosmertics), C12 15 alkyl benzoate (Making Cosmetics), Emullium Illustro (Gattefosse), bentone Gel ISD V (Elementis), microbead powder at the following weight concentrations, 12.5:12.5:5:3:5, respectively. Phase A is prepared by mixing the listed components. Phase B is prepared without the microbead powder by overhead stirring until all of the components are dissolved. Then Phase A is added to Phase B while stirring at 1000 rpm until fully mixed. Then the microbead powder is added to the mixture while mixing at 1000 rpm for 5 minutes. The resulting mixture is homogenized with an Ultra Turax for 5 minutes at 10,000 rpm. The haze transmission of drawdown films (38 um) after 5 min drying at 50° C. are measured using BYK Haze Gard I.

In one embodiment or in combination with any other embodiment mentioned herein, the hardened or jet milled CE microbeads exhibit a total transmission of at least 50, at least 60, at least 70, at least 75, at least 80, at least 85, at least 86, at least 87, at least 88, or at least 89 percent as measured using a BYK Haze-Gard I unit.

### Cosmetic Formulations

The hardened or jet milled CE microbeads produced by the processes disclosed herein may be used to produce a variety of cosmetic compositions. The cosmetic compositions may be produced by: (1) providing a plurality of the hardened or jet milled CE microbeads; (2) combining the hardened or jet milled CE beads with one or more cosmetic additives to thereby form a pre-cosmetic mixture; and (3) forming the cosmetic composition from the pre-cosmetic mixture.

In one embodiment or in combination with any other embodiment, the present application discloses a cosmetic composition comprising any of the CE microbeads (hardened or jet milled) disclosed herein.

In one embodiment or in combination with any other embodiment mentioned herein, the cosmetic composition can comprise at least 0.1, 0.5, 1, 2, 3, 4, 5, 6, 7, 8, 9, 10, 11, 12, 13, 14, or 15 weight percent of the hardened or jet milled CE microbeads. Additionally, or in the alternative, the cosmetic composition can comprise less than 99, 90, 80, 70, 60, 50, 40, 30, 25, 20, 15, 10, or 5 weight percent of the or jet milled hardened CE microbeads. For example, the cosmetic composition can comprise 0.1 to 90, 0.1 to 50, 0.1 to 30, 0.1 to 20, 0.1 to 15, 0.1 to 10, 0.1 to 5, 1 to 90, 1 to 50, 1 to 30, 1 to 20, 1 to 15, 1 to 10, or 1 to 5 weight percent of the hardened or jet milled CE microbeads.

In one embodiment or in combination with any other embodiment mentioned herein, the cosmetic composition can be a foundation, a sunscreen, a lipstick, a mascara, an eye shadow, a lotion, a dry shampoo, a liquid shampoo, a body wash, a lotion, a hair conditioner, a skin moisturizer, a face wash, a tablet, a foot powder, a baby powder, a shaving cream, or a shaving gel.

In one embodiment or in combination with any other embodiment mentioned herein, the cosmetic composition can be a loose powder, a compacted powder, a gel, an emulsion, a liquid, or an aerosol.

In one embodiment or in combination with any other embodiment mentioned herein, the cosmetic composition comprises at least 1, 5, 10, 15, 20, 25, 30, 35, 40, 45, 50, 55, 60, 65, 70, 75, 80, 85, 90, 95, or 99 weight percent of at least one, two, three, four, or five cosmetic additives. Additionally, or in the alternative, the cosmetic composition can comprise less than 99, 95, 90, 85, 80, 75, 70, 65, 60, 55, or 50 weight percent of at least one, two, three, four, or five cosmetic additives. For example, the cosmetic composition can comprise 1 to 99, 1 to 95, 1 to 90, 1 to 85, 1 to 80, 5 to 99, 5 to 95, 5 to 90, 5 to 85, 10 to 99, 10 to 95, 10 to 85, 10 to 80, 15 to 99, 15 to 95, 15 to 90, 15 to 85, or 15 to 80 weight percent of at least one, two, three, four, or five cosmetic additives.

Generally, the cosmetic additives can include a solvent, a colorant, an oil, a wax, a fatty acid, an alcohol, an ester, a hydrocarbon, a silicone oil, a surfactant, a metal soap, a moisturizer, a thickener, a UV absorber, an antioxidant, an oil absorbent, an exfoliant, water, or a combination thereof.

In one embodiment or in combination with any other embodiment mentioned herein, the colorant comprises a pigment (e.g., an organic pigment and/or an inorganic pigment) and/or a dye.

In one embodiment or in combination with any other embodiment mentioned herein, the oil comprises triglycine, soybean oil, cocoa butter, palm oil, palm kernel oil, hardened oil, and/or hardened castor oil.

In one embodiment or in combination with any other embodiment mentioned herein, the wax comprises carnauba wax, candelilla wax, lanolin, lanolin, candelilla wax, cotton wax, Montan wax, Kapok wax, lanolin acetate, lanolin, and/or lanolin fatty acid isopropyl.

In one embodiment or in combination with any other embodiment mentioned herein, the fatty acid comprises lauric acid, myristic acid, palmitic acid, stearic acid, isostearic acid, behenic acid, oleic acid, undecylenic acid, linoleic acid, eicosapentaenoic acid (EPA), and/or docosahexaenoic acid.

In one embodiment or in combination with any other embodiment mentioned herein, the alcohol comprises cetyl alcohol, stearyl alcohol, isostearyl alcohol, 2-octyldodecanol, lauryl alcohol, behenyl alcohol, myristyl alcohol, oleyl alcohol, and/or cetostearyl alcohol.

In one embodiment or in combination with any other embodiment mentioned herein, the ester comprises isopropyl myristate, 2-octyldodecyl myristate, cetyl 2-ethylhexanoate, diisostearyl malate, tripropylene glycol dineopentate, isononyl isononanoate, isotorideyl isononanoate, cetyl octanoate, isocetyl palmitate, butyl stearate, hexyl laurate, myristyl myristate, decyl oleate, hexyl decyl dimethyloctanate, cetyl lactate, myristyl lactate, lanolin acetate, isosetyl stearate, isosetyl isostearate, cholesteryl 12-hydroxystearate, di-2-ethylhexanoic acid ethylene glycol, dipentaerythritol fatty acid ester, monoisostearate N-alkylglycol, dicaprate neopentyl glycol, di-2-heptylundecanoate glycerin, tri-2-ethylhexanoate trimethylpropane, Trimethylolpropane triisostearate, pentaerythritol tetra-2-ethylhexanoate, glycerin tri-2-ethylhexanoate, glycerin trioctanoate, glycerin triisopalmitate, trimethylolpropane triisostearate, ethylhexyl palmitate, glycerin trimyristate, tri-2-heptylundecanoic acid glyceride, castor oil fatty acid methyl ester, oleyl oleate, acetoglyceride, 2-heptylundecyl palmitate, diisobutyl adipate, N-lauroyl-L-Glutamic hexyldecyl palmitate, adipate hexyldecyl, diisopropyl sebacate, ethylhexyl succinate, and/or triethyl citrate.

In one embodiment or in combination with any other embodiment mentioned herein, the hydrocarbon comprises paraffin, petrolatum, and/or microcrystalline wax.

In one embodiment or in combination with any other embodiment mentioned herein, the surfactant comprises an anionic surfactant, a cationic surfactant, and/or a nonionic surfactant.

In one embodiment or in combination with any other embodiment mentioned herein, the thickener comprises guar gum, pectin, starch, gelatin, collagen, cellulosic derivatives, and/or mannan.

### Experiments

### Abbreviations

CAB is cellulose acetate butyrate; DS_{OH} is average degree of substitution for hydroxyl substituents; DS_{Ac} is average degree of substitution for acetyl substituents; DS_{Bu} is average degree of substitution for butyryl substituents; PS means D(4, 3) particle size.

CE dopes were prepared as shown in Table 3 below to determine the solubility of CE, such as cellulose acetate butyrate (CAB), Example 1, in a solvent system containing ethyl acetate (EA), n-propanol (nPrOH), and water.

The preparation of Ex 1 is shown below.

### Example 1: Cellulose Acetate Butyrate (DS_{Ac}=1.87, DS_{Bu}=0.22, DS_{OH}=0.91 M_{w}=90766)

Cellulose and acid mixture [cellulose (4.3 parts) and AcOH (11.8 parts)] was added to an agitated reactor and soaked unheated then, the mixture was heated to 55° C. Sulfuric acid was added and the reaction mixture was cooled to 30° C. Following, a mixture (i.e., acylation solution) of Ac₂O (8.9 parts) and Bu₂O (5.6 parts) was added and the mixture cooled to around 9° C. with agitation. The resulting reaction mixture was warmed to 50° C. and the reaction mixture was stirred until the acylation was complete and the desired molecular weight was attained. To this reaction mixture was added a mixture of BuOH (18 parts) and H₂O (7.4 parts). The mixture was then stirred at 68° C. for 1020 min. The mixture was then quenched, precipitated in water, washed and dried by common methods.

### General Procedure for Preparation of Cellulose Esters

Cellulose mixture [cellulose and AcOH](“Cellulose Mixture”) and sulfuric acid, Ac₂O and Bu₂O (“Acylation Solution”) was cooled to 30° C. in an agitated reactor, and the reaction mixture cooled to around 7° C. with agitation. Additional sulfuric acid was added to a target amount, and the resulting reaction mixture, while stirring, was warmed to 45° C.-65° C. until the reagents were exhausted. The reaction mixture treated with aqueous AcOH and BuOH (“Hydrolysis Solution”) and stirred at 68° C. under the Hydrolysis Conditions. Then, the reaction mixture was then quenched, neutralized, precipitated, washed and dried by common methods.

Ex 2-5 were prepared by adapting the general procedure for the preparation of cellulose esters using the reagents and conditions shown in Table 1. Table 2 provides the various degrees of substitution and molecular weights for Ex 2-5.

TABLE 1

| | Cellulose | | | | | | | | | Hydrolysis | |
|---|---|---|---|---|---|---|---|---|---|---|---|
| | Mixture | | Acylation Solution | | | | Hydrolysis Solution | | | Conditions | |
| | Cellulose | AcOH | H₂SO₄ | AcOH | Ac₂O | Bu₂O | AcOH | BuOH | H₂O | Time | Temp |
| Ex | (parts) | (parts) | (parts) | (parts) | (parts) | (parts | (parts) | (parts) | (parts) | (min) | (° C.) |
| 2 | 43 | 167.5 | 3.1 | 3.1 | 89 | 56 | 184 | 84 | 146.2 | 720 | 68 |
| 3 | 43 | 167.5 | 3.1 | 3.1 | 89 | 56 | 184 | 84 | 146.2 | 720 | 68 |
| 4 | 43 | 167.5 | 3.1 | 3.1 | 89 | 56 | 184 | 84 | 146.2 | 840 | 68 |
| 5 | 43 | 167.5 | 3.1 | 3.1 | 89 | 56 | 184 | 84 | 146.2 | 1,020 | 68 |

TABLE 2

| Ex | Degrees of Substitution | MW |
|---|---|---|
| 2 | DS_{OH} = 0.6, DS_{Bu} = 0.21, DS_{Ac} = 2.19 | 109K |
| 3 | DS_{OH} = 0.7, DS_{Bu} = 0.21, DS_{Ac} = 2.08 | 68.3K |
| 4 | DS_{OH} = 0.8, DS_{Bu} = 0.2, DS_{Ac} = 1.99 | 72.2K |
| 5 | DS_{OH} = 0.9, DS_{Bu} = 0.2, DS_{Ac} = 1.86 | 78.2 |

### Degree of Substitution

The degree of substitution for the substituents on the cellulose ester backbone is calculated using proton nuclear magnetic resonance spectroscopy. Gel permeation chromatography is performed on cellulose esters in stabilized tetrahydrofuran. The instrument is an Agilent 1260 which consists of a degasser, isocratic pump with a flow rate of 1.0 milliliters per minute, autosampler with an injection volume of 25 microliters, a column oven set at 28° C. and a refractive index detector at 28° C. The column set consists of an Agilent PLgel 5 micron guard, Mixed-C and Oligopore in series. The system is calibrated with monodisperse polystyrene standard ranging from approximately 4 million to 162 molecular weight. The sample is prepared by weighing approximately 25 milligrams of sample in 10 milliliters of solvent with the addition of 10 microliters of toluene to be used as a flow rate marker, add a stir bar into an 8-dram screw cap vial and stir until dissolution.

### Molecular Weight

The molecular weight is determined by gel permeation chromatography. Gel permeation chromatography is performed on cellulose esters in stabilized tetrahydrofuran. The instrument is an Agilent 1260 which consists of a degasser, isocratic pump with a flow rate of 1.0 milliliters per minute, autosampler with an injection volume of 25 microliters, a column oven set at 28° C. and a refractive index detector at 28° C. The column set consists of an Agilent PLgel 5 micron guard, Mixed-C and Oligopore in series. The system is calibrated with monodisperse polystyrene standard ranging from approximately 4 million to 162 molecular weight. The sample is prepared by weighing approximately 25 milligrams of sample in 10 milliliters of solvent with the addition of 10 microliters of toluene to be used as a flow rate marker, add a stir bar into an 8-dram screw cap vial and stir until dissolution.

### Preparation of CE Dopes

The CE dopes were prepared by charging a dry, 250 mL, 1-neck round-bottomed flask equipped with a magnetic stirrer with the respective amount of the solvent systems. The solvent systems were stirred, and the flask was then charged with the respective amount/type of CAB. The CAB was charged into the flask by slowly metering the solids at a rate such that the stirring vortex was able to move the solid particles into the solvent system without forming a large mass of powder at the top of the liquid phase. This mixture of solvent and CAB was stirred at room temperature for 45 minutes. If the mixture became homogeneous in this time period, the Dope was determined to be “soluble.” If the solid particles remained undissolved in the solvent system at 60 minutes, the Dope was determined to be “insoluble.”

In Dopes 1-1, 1-2, and 1-3, the solvent system contained ethyl acetate and n-propanol in varying amounts, but did not contain water. In Dopes 1-1a, 1-2a, and 1-3a, water was added to the solvent system. That is, the solvent systems of Dopes 1-1 and 1-1a, 1-2 and 1-2a, and 1-3 and 1-3a contained the same amount of ethyl acetate and n-propanol, and the only difference between the respective Dopes was the addition of water.

In Dope 1-4a, the amount of CAB contained in the CE dope was increased to match its % CAB by mass to the % CAB by mass of the Dopes that did not contain water (i.e., Dopes 1-1, 1-2, and 1-3).

TABLE 3

| | EA | nPrOH | Water | CAB | EtOAc | nPrOH | Water | CAB | |
|---|---|---|---|---|---|---|---|---|---|
| Dope | (g) | (g) | (g) | (g) | (%) | (%) | (%) | (%) | Solubility |
| 1-1 | 80.0 | 5.0 | 0 | 15.0 | 80.0 | 5.0 | 0 | 15.0 | No |
| 1-1a | 80.0 | 5.0 | 8.0 | 15.0 | 74.1 | 4.6 | 7.4 | 13.9 | No |
| 1-2 | 75.0 | 10.0 | 0 | 15.0 | 75.0 | 10.0 | 0 | 15.0 | No |
| 1-2a | 75.0 | 10.0 | 8.0 | 15.0 | 69.4 | 9.3 | 7.4 | 13.9 | Yes |
| 1-3 | 70.0 | 15.0 | 0 | 15.0 | 70.0 | 15.0 | 0 | 15.0 | N0 |
| 1-3a | 70.0 | 15.0 | 8.0 | 15.0 | 64.8 | 13.9 | 7.4 | 13.9 | Yes |
| 1-4 | 70.0 | 15.0 | 8.0 | 16.2 | 64.8 | 13.9 | 7.4 | 15.0 | Yes |

Notably, CE dopes that did not contain water were unable to solubilize the CE contained therein. For example, Dope 1-1 provided a chunky and non-homogeneous mixture, Dopes 1-2 and 1-3 initially provided stirrable slurries but became chunky and non-homogeneous after several minutes.

In comparison, Dope 1-1a provided a stirrable, rather than chunky, slurry. Dopes 1-2a and 1-3a provided homogeneous mixtures in which the CE was dissolved in the solvent system within 3 minutes.

As shown in Dope 1-4a, increasing the mass of the CE contained in the CE dope also provided a homogenous mixture in which the CE was dissolved in the solvent system within 3 minutes. That is, increasing the % CAB of the CE dope did not appear to negatively impact the solubility of the CE in the solvent system.

### Emulsion Process for Preparation of Microparticles

A clean, dry 3-liter kettle flask (with baffles) was equipped with a two-tiered overhead mechanical agitator and bottom drop-out valve was prepared. The kettle was charged with 700-1,300 g deionized water, 6-13 g Tergitol 15-S-40, and 0-1.0 g PEG-100 stearate. Over 15 minutes, 7-12 g colloidal protector (low-, medium-, high-viscosity carboxymethylcellulose or methylcellulose) was added to the stirred aqueous mixture. The mixture was allowed to stir at ambient temperature for one hour or until homogeneous.

A separate clean, dry 2-liter kettle flask equipped with an overhead mechanical agitator and a bottom drop-out valve was prepared. A dope solution consisting of 10-100% cellulose ester was prepared using 0-5.0 parts C1-C4-alkyl acetate, 0-1.0 parts C1-C3 alcohol, and 0-0.80 parts deionized water (based on mass of the cellulose ester used). The mixture was allowed to stir at ambient temperature until homogeneous.

The dope solution was added to the 3-liter kettle over 30 minutes with vigorous stirring (250-500 rpm). Upon completion of the addition, the emulsion was recirculated through a flow cell containing a high-shear mixer (0-12,000 rpm) at the rate of 100-300 mL/minute for 45-60 minutes.

Upon completion of the high shear mixing time, the emulsion was then pumped at 300 mL/min into a 5-gallon bucket equipped with a single-tiered overhead mechanical agitator containing 4000-6000 g deionized water and agitated at 250-500 rpm for 0-16 hours. After the hold time, the volume was centrifuged to separate the spherical microparticles. The microparticles were suspended into 1-2 L of deionized water and centrifuged again. This washing process was repeated once.

The microparticles were placed in a sigma-bladed mixer and dried under vacuum (100-400 mmHg) at 50-100° C. for 16 hours. The typical recovered yield is 75-85%.

### Jet Milling Process for Preparation of Microparticles

There are multiple jet-milling configurations that can be used to reduce the size of particles. Such configurations are discussed in A. Chamayou and J. A. Dodds, Air Jet Milling, Handbook of Powder Technology, volume 12, Chapter 8, 2007 (“Chamayou”). FIG. 7 of Chamayou provides an example of a fluidized bed opposed jet mill that can be used to reduce the size of the cellulose ester particles described below in Table 4. The jet-milling process was used to reduce cellulose ester average particle size from 300-900 μm to ˜10 μm. Fluidized bed opposed jet mills operates as follows: The cellulose ester is placed into a hopper and introduced into the top of the mill (“FEED IN”) typically though a double valve arrangement (or through an injector). The cellulose ester particles fall by gravity to the bottom of the mill where they are swept up into one of three high pressure air streams that are geometrically oriented towards one another thus forming the so-called “pulverizing zone.” Within the pulverizing zone, the cellulose ester particles are size reduced via interparticle collisions. The size-reduced particles are then conveyed upwards by mass transport in the vertical airstream (fluidized bed) ultimately carrying them into the classifier. The classifier allows particles below the desired minimum size to be removed from the mill (“FINE OUT”). Particles that are above the maximum size are excluded from the classifier and returned to the fluidized bed eventually falling back down into the pulverizing zone for further size reduction. Particles that fall within the desired size range are ejected from the classifier into an appropriate product container. Many control parameters exist for optimizing productivity, particle size and particle size distribution shape, including but not necessarily limited to, classifier rotor speed, air nozzle pressure, and bed level.

TABLE 4

| Ex # | Microparticle | Material | Description |
|---|---|---|---|
| 2-1 | Milled CE | Ex 2 | Prepared by jet mill, Spheroid shape, |
| | Microparticles | | D(10) = 3.85 μm, D(50) = 6.81 μm, D(90) = |
| | | | 11.5 μm, Span = 1.1, Certified >60% |
| | | | biodegradable under OECD 301F |
| 3-1 | Milled CE | Ex 3 | Prepared by jet mill, spheroid shaped, |
| | Microparticles | | D(10) = 3.15 μm, D(50) = 6.4 μm, D(90) = |
| | | | 11.3 μm, Span = 1.3, Certified >60% |
| | | | biodegradable under OECD 301F |
| 4-1 | Emulsion based | Ex 4 | Prepared by emulsion process, spherically |
| | CE Microparticles | | shaped, D(10) = 5.86 μm, D(50) = 11.3 μm, |
| | | | D(90) = 21.1 μm, Span = 1.1, Certified >60% |
| | | | biodegradable under OECD 301F |
| 4-2 | Emulsion based | Ex 4 | Prepared by emulsion process, spherical |
| | CE microparticles | | shaped, D(10) = 6.49 μm, D(50) = 14.6 μm, |
| | | | D(90) = 28.7 μm, Span = 1.5 |
| 5-1 | Milled CE | Ex 5 | Prepared by jet mill, spheroid shape, |
| | Microparticles | | D(10) = 3.16 μm, D(50) = 6.47 μm, D(90) = |
| | | | 11.8 μm, Span = 1.3 |
| CEx 6 | KOBO SP-10 | Nylon-12 | — |
| CEx 7 | KOBO MSP-822 | Polymethyl | — |
| | | Methacrylate | |
| CEx 8 | KOBO GWC-500EP | Polymethylsilse | — |
| | | squioxane and | |
| | | Silica | |
| CEx 9 | Sunsphere H-53 | Silica | — |
| CEx 10 | Dimethicone 500 | Dimethicone | — |

### OECD 301F Test Description

In this experiment, a measured volume of a mineral medium is inoculated with a known concentration of the test substance, which serves as nominal source of organic carbon. The medium is placed in a closed flask and stirred at a constant temperature (within a range of +1° C. or closer) for a maximum of 60 days.

The consumption of oxygen is determined using one of two methods: either by measuring the amount of oxygen required (produced electrolytically) to maintain a constant gas volume in the respirometer flask, or by monitoring changes in volume or pressure (or a combination of both) in the apparatus.

Any carbon dioxide produced during the process is absorbed using a solution of potassium hydroxide or another suitable absorbent. The amount of oxygen utilized by the microbial population during the biodegradation of the test substance is calculated by subtracting the oxygen uptake by the blank inoculum (which runs in parallel). This value is expressed as a percentage of Theoretical Oxygen Demand (ThOD) or, less ideally, Chemical Oxygen Demand (COD).

### Haze Measurements

Haze percentage of the formulations were measured using the BYK haze gard. The haze gard instrument creates a light beam that strikes the sample where a portion of the light is transmitted and enters an integrating sphere measuring transmission haze. It uses unidirectional illumination and diffuse viewing.

### Haze Measurement Procedure

Clean gorilla glass with acetone or ethyl alcohol to ensure there is no streaking on substrate. Draw down the formulation on the gorilla glass using a 1.5-mil square drawdown bar. Pull the bar from top to bottom at constant speed/pressure down the substrate to make a uniform sample for testing. Samples should be uniform and of equal thickness. Place sample in oven to dry for 5 minutes at 35-40° C. Let sample completely cool after removing from oven. Place the draw down sample in front of the haze sensor for haze measurements. Take three readings on one sample at top, middle, and bottom of drawdown. Perform 3 drawdowns per sample. Average the nine readings and report haze percentage.

### Haze of Microparticles in Single Oil Dispersions

Table 5 provides the haze of various microparticles in single oil dispersions. The dispersions were either 5 wt % or 7 wt % in dimethicone. A mixer was used to fully disperse the microparticles in the dimethicone.

All of microparticles added showed higher haze numbers compared to dimethicone oil base. Cellulose ester microparticles showed similar or better haze % compared to external benchmark microparticles. By demonstrating higher haze %, it is concluded that cellulose ester microparticles imparted enhanced optical effects.

TABLE 5

| | |
|---|---|
| 5 wt % | 7 wt % |
| Dispersion | Dispersion |

| Ex # | T (%) | Haze (%) | T (%) | Haze (%) |
|---|---|---|---|---|
| 2-1 | 93.9 | 22.0 | 93.9 | 30.0 |
| 3-1 | 93.7 | 25.6 | — | — |
| 4-1 | 93.8 | 23.2 | 93.7 | 37.5 |
| 4-1 | 92.7 | 20.2 | 91.9 | 28.9 |
| 5-1 | 94.1 | 23.5 | 93.9 | 27.5 |
| CEx 6 | 93.8 | 17.8 | 93.8 | 22.6 |
| CEx 7 | 93.6 | 35.1 | 93.6 | 41.3 |
| CEx 8 | 94.5 | 4.0 | 93.5 | 41.3 |
| CEx 9 | 94.0 | 8.4 | 94.5 | 2.5 |
| CEx 10 | 94.6 | 0.1 | 94.6 | 0.1 |

1. Biodegradable microparticles comprising a mixed cellulose ester,
wherein the biodegradable microparticles exhibit at least 50 percent biodegradability at 60 days according to at least one of the OECD 301B, OECD 301C, or OECD 301F test methods, and a haze transmission of at least 10 percent,
wherein the mixed cellulose ester comprises-
(a) an average degree of substitution for acetyl substituents (“DS_{Ac}”) in the range of 0.1 to 2.3,
(b) an average degree of substitution for propionyl substituents (“DS_{Pr}”) or an average degree of substitution for butyryl substituents (“DS_{Bu}”) in the range of 0.1 to 1.5, and
(c) an average degree of substitution for hydroxyl substituents (“DS_{OH}”) in the range of 0.6 to 2.8.

2. The biodegradable microparticles according to claim 1, wherein the biodegradable microparticles exhibit a haze transmission of at least 15, at least 20, at least 25, at least 30, at least 35, at least 40, or at least 45 percent, and/or wherein the biodegradable microparticles exhibit a haze transmission of less than 90, or less than 85, or less than 80, or less than 75, or less than 70, or less than 65, or less than 60, or less than 55, or less than 50, or less than 45, or less than 40 percent, or less than 35 percent, or less than 32 percent, or less than 30 percent as measured using a BYK Haze-Gard I unit.

3. The biodegradable microparticles according to claim 1, wherein the biodegradable microparticles exhibit a monomodal particle size distribution with a span of at least 0.5.

4. The biodegradable microparticles according to claim 1, wherein the biodegradable microparticles have a D50 volume-based particle size in the range of 5 to 20 microns.

5. The biodegradable microparticles according to claim 1, wherein the biodegradable microparticles exhibit at least 40 percent biodegradability at 60 days according to at least one of the OECD 301B, OECD 301C, or OECD 301F test methods; and/or wherein the mixed cellulose ester exhibits at least 40, or at least 45, or at least 50, or at least 55, or at least 60, or at least 65, or at least 70, or at least 75, or at least 80, or at least 85, or at least 90, or at least 95 percent biodegradability at 60 days according to at least one of the OECD 301B, OECD 301C, or OECD 301F test methods.

6. The biodegradable microparticles according to claim 1, wherein the DS_{Ac} is at least 0.2.

7. The biodegradable microparticles according to claim 6, wherein the DS_{Ac} is less than 2.3.

8. The biodegradable microparticles according to claim 1, wherein the DS_{Pr} is at least 0.2.

9. The biodegradable microparticles according to claim 8, wherein the DS_{Pr} is less than 1.5.

10. The biodegradable microparticles according to claim 1, wherein the DS_{Bu} is at least 0.2.

11. The biodegradable microparticles according to claim 1, wherein the DS_{OH} is at least 0.6.

12. The biodegradable microparticles according to claim 1, wherein the mixed cellulose ester is a cellulose acetate butyrate, or a cellulose acetate butyrate, or a mixture thereof.

13. The biodegradable microparticles according to claim 12, wherein the mixed cellulose ester is a cellulose acetate butyrate.

14. The biodegradable microparticles according to claim 1, wherein the biodegradable microparticles comprise at least 50 weight percent of the mixed cellulose ester.

15. The biodegradable microparticles according to claim 1, wherein the biodegradable microparticles consist essentially of the mixed cellulose ester.

16. The biodegradable microparticles according to claim 1, wherein the biodegradable microparticles have a sphericity of at least 10.

17. The biodegradable microparticles according to claim 1, wherein the biodegradable microparticles exhibit an oil absorption of at least 30 mL per 100 g at least 35 mL per 100 g, as measured using test method ASTM D281, wherein mineral oil is used instead of castor oil.

18. A process for forming biodegradable microparticles, the process comprising:
(a) forming a dope comprising a mixed cellulose ester, wherein the mixed cellulose ester comprises-
(i) an average degree of substitution for acetyl substituents (“DS_{Ac}”) in the range of 0.1 to 2.3,
(ii) an average degree of substitution for propionyl substituents (“DS_{Pr}”) or an average degree of substitution for butyryl substituents (“DS_{Bu}”) in the range of 0.1 to 1.5, and
(ii) an average degree of substitution for hydroxyl substituents (“DS_{OH}”) in the range of 0.6 to 2.8;
(b) contacting at least a portion of the dope with an aqueous mixture under agitation to thereby form an initial emulsion; and
(c) converting at least a portion of the initial emulsion into a dispersion comprising a solid phase and a liquid phase, wherein the solid phase comprises biodegradable microparticles; and
(d) recovering at least a portion of the biodegradable microparticles from the dispersion, wherein the biodegradable microparticles exhibit at least 50 percent biodegradability at 60 days according to at least one of the OECD 301B, OECD 301C, or OECD 301F test methods, wherein the biodegradable microparticles exhibit a haze transmission of at least 10 percent.

19. The process according to claim 18, wherein the biodegradable microparticles exhibit a haze transmission of at least 15, or at least 20, or at least 25, or at least 30, or at least 35, or at least 40, or at least 45 percent, and/or wherein the biodegradable microparticles exhibit a haze transmission of less than 90, or less than 85, or less than 80, or less than 75, or less than 70, or less than 65, or less than 60, or less than 55, or less than 50, or less than 45, or less than 40 percent, or less than 35 percent, or less than 32 percent, or less than 30 percent.

20. The process according to claim 18, wherein the biodegradable microparticles exhibit a monomodal particle size distribution with a span of at least 0.5; and/or
wherein the biodegradable microparticles have a D50 volume-based particle size in the range of 1 to 100 microns.

21. The process according to claim 18, wherein the biodegradable microparticles exhibit at least 40 percent biodegradability at 60 days according to at least one of the OECD 301B, OECD 301C, or OECD 301F test methods, and/or wherein the mixed cellulose ester exhibits at least 40 percent biodegradability at 60 days according to at least one of the OECD 301B, OECD 301C, or OECD 301F test methods.

22. The process according to claim 18, wherein the DS_{OH} is at least 0.6.

23. The biodegradable microparticles according to claim 18, wherein the mixed cellulose ester is a cellulose acetate butyrate.

24. The biodegradable microparticles according to claim 18, wherein the biodegradable microparticles have a sphericity of at least 10 percent, and/or wherein the biodegradable microparticles have a sphericity of less than 100 percent.

25. The process according to claim 18, wherein the biodegradable microparticles exhibit an oil absorption of at least 30 mL per 100 g at least 35 mL per 100 g, as measured using test method ASTM D281, wherein mineral oil is used instead of castor oil.

26. A cosmetic composition comprising the biodegradable microparticles of claim 1.
